(19) United States
(12) Patent Application Publication
(10) Pub. No.: US 2005/0165284 A1
(43) Pub. Date: Jul. 28, 2005

(54) Method and system for determining a risk of ulcer onset

(76) Inventor: Amit Gefen, Ganei Tikva (IL)

Correspondence Address: Martin Moynihan;Anthony Castorina, 2001 Jefferson Davis Highway, Suite 207, Arlington, VA, 22202, US

(21) Appl. No.: 10/508,002

(22) PCT Filed: Mar. 10, 2003
(86) PCT No.: PCT/IL03/00193

(30) Foreign Application Priority Data
Mar. 25, 2002 (US) 60366588
Aug. 2, 2002 (US) 60400133
Oct. 30, 2002 (US) 60422118

(51) Int. Cl. A61B 5/00; A61B 19/00

(52) U.S. Cl. 600/300; 600/549; 600/547; 600/557

(57) ABSTRACT

A system for determining a risk of pressure ulcer onset on a subject being in contact with a supporting-surface, the system comprising: an arrangement of sensors, located at predetermined locations between the supporting-surface and the subject; and a data processor for receiving and processing data sensed by the sensors; the sensors and the data processor being designed and programmed for determining the risk of pressure ulcer onset on the subject being in contact with the supporting-surface.

### FIELD AND BACKGROUND OF THE INVENTION

The present invention relates to a method and a system for determining a risk of ulcer onset and, more particularly, to a method and a system for measuring the temperature of and the load on an organ over time, thereby to determine the risk of developing ulcers, such as pressure sores and the like.

Hospitalized patients, immobile individuals, bed-ridden or wheelchair bounded individuals, anesthetized patients undergoing prolonged surgery and others who may be required to spend extended periods of time in either a sitting or prone position often suffer muscle, subcutaneous, skin and other soft tissue necrosis caused by prolonged pressure. Tissue necrosis develops when there is a lack of oxygen and other nutrients needed to satisfy the metabolic demands of the tissue, due to a partial or total occlusion of blood vessels. One example of tissue necrosis pressure ulcers also known as pressure sores, bed sores and decubitus ulcers.

In a necrosis state, the lymphatic and venous drainage are also impaired and the metabolic wastes are left to accumulate in the damaged tissue. Any degree of local occlusion of blood supply could lead to anaerobic respiration if oxygen supply does not meet demand. Lactate production would then rise as a consequence of increased glycolytic rate [Knight L. S., Taylor P. R., Polliac A. A. & Bader L. D. (2001) “Establishing predictive indicators for the status of loaded soft tissues”, Journal of Applied Physiology, 90: 2231-2237]. As energy stores diminish, the tissue begins to damage and cells begin to die. Any relief of the pressure, before necrosis proceeds, allows nutrients back into the area and waste products to be carried off.

Normally, subjects are supported by surfaces such as chairs and/or beds, and the bodies press against the skin surface with a total force generally equal to the body weight. The physiology of the human body is such that, when the body rests on a support there will always be certain body portions subject to elevated pressure relative to other body portions. Should such peak local pressure be maintained for a prolonged period, and not be relieved by changing the posture of lying or sitting, pressure ulcers are very likely to developed.

The development of pressure ulcers is aggravated by excessive temperature and perspiration. Normal, body-able individuals will start to feel pain when subjected to continuous local pressure and will therefore shift their body automatically to lessen the discomfort, but patients having a sensory loss (as a result of, for example, vascular damage or neural damage, or while being in an unconscious state) are deprived of this protection and are therefore most vulnerable to pressure ulcers. The severity of the formed pressure ulcers depends on many subject-specific parameters such as age, nutritional status, body structure, general health condition and medication and even psychosocial state.

Although some pressure ulcers may eventually heal, the time scale for such healing is rather long and depends on the severity of the damage. However, the muscles and skin do not fully recover even after healing. In addition, scarring and tissue loss following a pressure ulcer considerably increase future risk for re-ulceration.

FIG. 1 illustrates a portion of a skin (epidermis and dermis), a portion of a subcotaneous tissue underneath the skin and a portion of a muscle tissue underneath the subcotaneous tissue. The development of pressure ulcers is commonly divided into four stages [Shea J. D. (1975), “Pressure sores: classification and management”, Clinical Orthopedics and Related Research, 112: 89-100], depending on the depth of the damage to the skin and underlying tissue, as follows:

With reference to FIG. 1, in stage I, the skin is intact and discoloration starts to occur. Stage I is sometime accompanied by warmth or hardness of the skin. If treated with the suitable dressing, a Stage I pressure ulcer is likely to heal within a few weeks.

Stage II is characterized by partial thickness skin loss or damage involving the epidermis and oftentimes the dermis. The ulcer is superficial and presents clinically as an abrasion, blister or shallow crater. With a proper treatment, a Stage II pressure ulcer could heal within 2-4 months.

Stage III is characterized by a full thickness skin loss involving damage of subcutaneous tissue but not extending to the underlying tissue. The ulcer presents clinically as a deep crater with or without undermining of adjacent tissue. Healing of a Stage III pressure ulcer may take several months.

In Stage IV there is a full thickness skin loss with extensive destruction, tissue necrosis, or damage to muscle, bone, or supporting structures (e.g., tendon, joint capsule). Healing of a Stage IV pressure ulcer, if at all possible, may take from several months to several years [Lewis B. (1996), “Protein levels and the etiology of pressure sores”, Journal of Wound Care, 4: 77-83]. In some severe cases of pressure sores, loss of limbs or even mortality due to blood infection may result.

Muscle tissue is highly vascularized and, therefore, is vulnerable to nutrient deprivation due to mechanical load or surface pressure. Pressure is defined as the applied force per unit of area, specifically, the smaller the area over which a given force is applied, the larger the pressure is. Thus, the most common sites for formation of pressure ulcer are in the tissue overlying bony prominences, where a small area of muscle or other deep tissue is under load [Welch C. B. (1990), “Preventing pressure sores”, British Medical Journal, 300: 1401-1404]. In particular, about 80% of pressure ulcers occur under the sacrum, ischium, trochanter and heel [Dealy C. (1991), “The size of the pressure sore problem in a teaching hospital”, Journal of Advanced Nursing, 16: 633-670], where an interface pressure of 40 to 100 mmHg was measured [Thompson-Bishop J Y. and Mottola C M. (1992), “Tissue interface pressure and estimated subcutaneous pressure of 11 different pressure-reducing supporting-surfaces”, Decubitus, 5: 42-48]. In infants below 18 month of age, however, pressure ulcers are developed on the occiput.

The time-frame for occurrence of pressure ulcers depends on various factors. One factor is, of course, the pressure. If the pressure exerted on the tissue is higher than the capillary pressure, the vessels collapse and the flow in the capillaries and lymph nodes reduces, causing an insufficient supply of oxygen and nutrients as well as insufficient disposal of metabolic wastes in the tissue. The loss of circulation results in a damage to the tissue. Both the duration of the pressure, and its intensity, plays a role in the development of pressure ulcers. For example, it has been shown [Kosiak M. (1961), “Etiology of decubitus ulcers”, Archives of Physical Medicine and Rehabilitation, 42: 19-29] that application of low pressure (70 mmHg) for two hours produced ischemic changes, while application of high pressure (190 mmHg) for one hour showed no microscopic changes.

A second factor is shearing forces which may be present. Shearing forces act as pairs of forces each applied on a specific object in an opposite direction, causing a distortion of the object. These pairs are applied to the skin when a bone pulls the skin in one direction while the supporting-surface applies a friction in the opposite direction. For example, if the patient is in a half-sitting position (recumbency), the gravitation force acting on the bone downward while the friction force acts on the external surface of the skin upwards. The result of shearing forces is a weakening of the skin and interruption of blood flow by distortion of the vascular network [Goossens R. H. M., et al. (1994), “The influence of shear on skin oxygen tension”, Clinical Physics and Physiological Measurement, 14: 111-118]. This makes the skin more vulnerable to the effects of direct pressure. It has been found [Rowland J. (1993), “Pressure ulcers. A literature review and a treatment scheme”, Australian Family Physician, 22: 1819-1827] that in the presence of enough shearing force, only half the pressure is required to obtain vascular occlusion.

A third factor is the temperature, which is closely related to the development of pressure ulcers. A small amount of temperature increment (of the order of 1° C.) causes an increased metabolic activity and oxygen demand. Hence for higher temperature the pressure threshold which is sufficient for the formation of pressure ulcers is smaller. On the other hand, heat loss may cause itself vasoconstriction and discomfort to the patient [Mahanty S D. and Roemer R B. (1980), “Thermal response of skin to the application of localized pressure”, Archives of Physical Medicine and Rehabilitation, 60: 584-590; Maklebust J. (1987), “Pressure ulcers: etiology and prevention”, Nursing Clinics of North America, 22: 359-377].

A fourth factor is the moisture. Frequent or excessive contact with moisture may reduce the tensile strength of the skin, resulting in skin breakdown [Longe R L. (1986), “Current concepts in clinical therapeutic: pressure sores”, Clinical pharmacology & therapeutics, 669-681]. Many studies have indicated that moist skin is more susceptible to injury from friction and more easily abraded (to this end see, e.g., Zimmerer R E., et al. (1986) “The effects of wearing diapers on skin”, Pediatric Dermatologist, 3: 95-101). Local skin temperature of above 38° C. is most likely to cause a local sweat response [Brengelmann G. L., Savage M. V., Avery D. H. (1994), “Reproducibility of core temperature threshold for sweating onset in humans”, Journal of Applied Physiology, 77: 1671-1677] and thereby induce a moist environment.

Pressure ulcers are painful and, as stated, slow, difficult to heal and in severe cases they may require amputation or cause death [Smith P. W., Black J. M., Black S. B. (1999) “Infected pressure ulcers in the long-term-care facility”, Infect Control Hosp Epidemiol 20: 358-61]. Until recently, pressure ulcers have been regarded as an inevitable outcome of aging and nursing home confinement. Nowadays, however, numerous efforts are devoted to studying the etiology of pressure ulcers, and their development is regarded as highly preventable. The most common and reasonable solution to the problem is to reduce the formation of such sores by regularly moving the patient in order to prevent the continuous application of the pressure to the discrete and poorly sensitive-to-pain surfaces of his body.

One procedure for preventing pressure ulcers is by implanting carbon pads over the ischial tuberosities to increase the load-bearing area over the ischium thereby to decrease pressure [Sutton R A. et al. (1987), “Soft tissue augmentation as a method of reducing the liability to pressure sores in spinal injured patients”, Paraplegia, 25: 454-465]. The dramatic invasiveness of this procedure, however, makes it impractical and not suitable for most patients.

Most of the techniques for minimizing pressure ulcers are directed at the development of supporting-surfaces. In a study made in 1996 by Collier M. E. entitled “Evaluation: pressure-reducing mattresses”, and published in Journal of Wound Care, 5: 207-211, eight pressure-relieving mattresses were compared with standard hospital mattress. The study concluded that such pressure-relieving mattresses provide more comfort and better tissue interface pressure. Improved supporting-surfaces which are presently known are filled with air, foam, fiber, water, gel and beads.

However, it is recognized that an improved support for the patient provide only a partial solution for the problem of pressure ulcers formation, and immobilized subjects, unless regularly moved from one position to another, are still at a substantial risk of developing pressure ulcers.

Other types of solutions are dynamic supporting-surfaces. For example, one improved supporting-surface is made out of continuously inflating and deflating cells, which allow for changes in the interface pressure thereby temporarily relieving sections of the body from pressure and creating a pressure gradient that may enhance blood flow and lymph drainage. These types of devices provide pressure relief on a 5-10 minute cyclic basis, without disturbing the patient. Another type of dynamic supporting-surface is an electric bed which rolls the patient from side to side.

Dynamic solutions, however, are rather expensive. The estimated daily cost for using a dynamic supporting-surface is from 40 to 90 US dollars.

Many attempts have been made to understand the formation of pressure ulcers, which attempts were primarily directed at the development of skin necrosis. However, the complex and multi-dimensional mechanisms underlying the etiology of pressure ulcers are not yet fully understood.

The present invention provides solutions to the problems associated with prior art techniques of preventing pressure ulcers.

### SUMMARY OF THE INVENTION

According to one aspect of the present invention there is provided a system for determining a risk of pressure ulcer onset on a subject being in contact with a supporting-surface, the system comprising: an arrangement of sensors, located at predetermined locations between the supporting-surface and the subject; and a data processor for receiving and processing data sensed by the sensors; the sensors and the data processor being designed and programmed for determining the risk of pressure ulcer onset on the subject being in contact with the supporting-surface.

According to further features in preferred embodiments of the invention described below, the system further comprising the supporting-surface, wherein the arrangement of sensors is integrated within or on the supporting-surface.

According to still further features in the described preferred embodiments the system further comprising a risk parameter calculator for calculating a risk parameter, α, characterizing the risk of the ulcers onset.

According to still further features in the described preferred embodiments the risk parameter calculator is operable to calculate at least one dose index, and further wherein α is calculated using the at least one dose index.

According to still further features in the described preferred embodiments the risk parameter calculator is operable to calculate a plurality of dose indices.

According to still further features in the described preferred embodiments the plurality of dose indices are selected from the group consisting of a pressure dose index, PDI, a temperature dose index, TDI and a humidity dose index, HDI.

According to still further features in the described preferred embodiments the risk parameter calculator further comprises at least one integrator for obtaining the at least one dose index.

According to still further features in the described preferred embodiments the risk parameter calculator comprises at least one normalizer for normalizing at least a portion of the at least one dose index, using a respective reference dose.

According to still further features in the described preferred embodiments the risk parameter calculator comprises an interfacial stress converter, for converting an interfacial stress, transmitted from the sensors, into an internal stress, using at least one conversion function.

According to still further features in the described preferred embodiments the risk parameter calculator comprises a first integrator for integrating the internal stress over a first predetermined period, thereby to obtain the PDI.

According to still further features in the described preferred embodiments the risk parameter calculator comprises a pressure normalizer for normalizing the PDI, using a reference pressure dose.

According to still further features in the described preferred embodiments the risk parameter calculator comprises a second integrator for integrating a temperature transmitted from the sensors over a second predetermined period, thereby to obtain the TDI.

According to still further features in the described preferred embodiments the risk parameter calculator comprises a temperature normalizer for normalizing the TDI using a reference temperature dose.

According to still further features in the described preferred embodiments the risk parameter calculator comprises a third integrator for integrating a humidity over a third predetermined period, thereby to obtain the HDI.

According to still further features in the described preferred embodiments the risk parameter calculator comprises a humidity normalizer for normalizing the HDI using a reference humidity dose.

According to still further features in the described preferred embodiments the sensors and the data processor are designed and programmed to alert prior to a formation, on the subject, of at least one ulcer adjacent to at least one of the sensors.

According to still further features in the described preferred embodiments the alert is produced if α is above a predetermined threshold.

According to still further features in the described preferred embodiments the alert is selected from the group consisting of an audio-alert, a visual-alert and a combination of an audio-alert and a visual-alert.

According to still further features in the described preferred embodiments the system further comprising a display for displaying a risk status for each of the predetermined locations.

According to still further features in the described preferred embodiments the system further comprising an A/D card.

According to still further features in the described preferred embodiments the system further comprising a communication channel for transmitting information from the system to a remote location.

According to still further features in the described preferred embodiments the communication channel is designed connectable to a telemetry apparatus.

According to still further features in the described preferred embodiments the communication channel is designed connectable to a telemedicine apparatus.

According to still further features in the described preferred embodiments the system further comprising memory media, storing in a retrievable and/or displayable format a risk status for each of the predetermined locations.

According to another aspect of the present invention there is provided a method of determining a risk of pressure ulcer onset on a subject being in contact with a supporting-surface, the method comprising: inputting information from an arrangement of sensors, located on predetermined locations between the supporting-surface and the subject; using the information for determining the risk of pressure ulcer onset on the subject being in contact with the supporting-surface.

According to further features in preferred embodiments of the invention described below, the method further comprising calculating a risk parameter, α, characterizing the risk of the ulcers onset.

According to still further features in the described preferred embodiments the calculation of α is by calculating at least one dose index.

According to still further features in the described preferred embodiments calculation of α is by calculating a plurality of dose indices.

According to still further features in the described preferred embodiments the plurality of dose indices are selected from the group consisting of a pressure dose index, PDI, a temperature dose index, TDI and a humidity dose index, HDI.

According to still further features in the described preferred embodiments α is a combination of at least two of the plurality of dose indices.

According to still further features in the described preferred embodiments the method further comprising normalizing at least a portion of the at least one dose index, using a respective reference dose.

According to still further features in the described preferred embodiments the method further comprising, for each of the predetermined locations, integrating a temperature measured at a respective location, over a second predetermined period, thereby obtaining the TDI.

According to still further features in the described preferred embodiments the method further comprising alerting prior to a formation, on the subject, of at least one ulcer adjacent to at least one of the sensors.

According to still further features in the described preferred embodiments the alerting is if α is above a predetermined threshold.

According to still further features in the described preferred embodiments the alerting is selected from the group consisting of audio-alerting, visual-alerting and a combination of audio-alerting and visual-alerting.

According to still further features in the described preferred embodiments the method further comprising displaying a risk status for each of the predetermined locations.

According to still further features in the described preferred embodiments the method further comprising storing a risk status for each of the predetermined locations, in a retrievable and/or displayable format on memory media.

According to yet another aspect of the present invention there is provided a method of characterizing a risk of ulcer onset the method comprising calculating a plurality of dose indices and a risk parameter, α, which is a combination of the plurality of dose indices, thereby characterizing the risk of the ulcer onset.

According to still another aspect of the present invention there is provided a method of preventing ulcers to be formed on a subject, the method comprising: calculating a plurality of dose indices and a risk parameter, α, which is a combination of the plurality of dose indices; and if a is above a predetermined threshold, then changing a position of the subject.

According to an additional aspect of the present invention there is provided a method of preventing ulcers to be formed on a subject being in contact with a supporting-surface, the method comprising: inputting information from an arrangement of sensors, located on predetermined locations between the supporting-surface and the subject; using the information for calculating a plurality of dose indices and a risk parameter, α, which is a combination of the plurality of dose indices; and if α is above a predetermined threshold, then changing a position of the subject.

According to further features in preferred embodiments of the invention described below, the plurality of dose indices are selected from the group consisting of a pressure dose index, PDI, a temperature dose index, TDI and a humidity dose index, HDI.

According to still further features in the described preferred embodiments the combination of the at least two of the plurality of dose indices is selected from the group consisting of a multiplication, a convolution, a power-law combination and a linear combination.

According to still further features in the described preferred embodiments α is calculated using a logic decision procedure.

According to still further features in the described preferred embodiments the risk parameter calculator is operable to calculate a using a logic decision procedure.

According to still further features in the described preferred embodiments the arrangement of sensors is designed locatable on a supporting-surface selected from the group consisting of a bed, a mattress, a chair, a wheelchair, an armchair, an operating table and a surface of a prosthesis being in contact with a residual limb.

According to still further features in the described preferred embodiments at least a portion of the arrangement of sensors is integrated within or on a clothing of the subject.

According to still further features in the described preferred embodiments the method further comprising converting an interfacial stress into an internal stress, using at least one conversion function.

According to still further features in the described preferred embodiments the at least one conversion function is selected from the group consisting of a polynomial conversion function, an exponential conversion function, a rational conversion function, a power conversion function, a look-up table and any combination thereof.

According to still further features in the described preferred embodiments the at least one conversion function varies with at least one parameter selected from the group consisting of a weight, a body structure, an organ geometry, an age, a nutritional status, a general health condition, medications administered to the subject, a tissue mechanical property, a geometry of the supporting surface and a mechanical property of the supporting surface.

According to still further features in the described preferred embodiments the tissue mechanical property is selected from the group consisting of a Young modulus, a Poisson's ratio, a shear modulus, a bulk modulus, a Lamé coefficients, a tangent elastic modulus, a plurality of hyper-elastic material model invariants and a plurality of coefficients which characterize a phenomenological function describing experimental stress-strain data.

According to still further features in the described preferred embodiments the tissue mechanical property is selected from the group consisting of a dimensionless mechanical property and a dimensionfull mechanical property.

According to still further features in the described preferred embodiments the method further comprising integrating the internal stress over a first predetermined period, thereby obtaining the PDI.

According to still further features in the described preferred embodiments the method further comprising normalizing the PDI, using a reference pressure dose.

According to still further features in the described preferred embodiments the reference pressure dose is selected so that if the PDI exceeds a unity then the subject is substantially at risk of the ulcer onset at a respective location.

According to still further features in the described preferred embodiments the method further comprising integrating a temperature over a second predetermined period, thereby obtaining the TDI.

According to still further features in the described preferred embodiments the method further comprising normalizing the TDI using a reference temperature dose.

According to still further features in the described preferred embodiments the reference temperature dose is selected so that if the TDI exceeds a unity, then the subject is substantially at risk of the ulcer onset at a respective location.

According to still further features in the described preferred embodiments the method further comprising integrating a humidity over a third predetermined period, thereby to obtain the HDI.

According to still further features in the described preferred embodiments the method further comprising normalizing the HDI using a reference humidity dose.

According to still further features in the described preferred embodiments α is a combination of the PDI, the TDI and the HDI.

According to still further features in the described preferred embodiments the method further comprising transmitting information to a remote location.

According to still further features in the described preferred embodiments the remote location is a nursing control center.

According to still further features in the described preferred embodiments the transmitting information is via a telemetry apparatus.

According to still further features in the described preferred embodiments the transmitting information is via a telemedicine apparatus.

According to yet an additional aspect of the present invention there is provided a device for determining contact parameters of a subject being in contact with a supporting-surface, the device comprising clothing, an arrangement of sensors and at least one communication channel for transmitting information sensed by the arrangement of sensors, the arrangement of sensors being integrated within or on the clothing, at predetermined locations.

According to further features in preferred embodiments of the invention described below, the contact parameters are selected from the group consisting of stress, pressure, temperature and humidity.

According to still further features in the described preferred embodiments each of the sensors is of a configuration designed not to substantially influence the risk of the subject at developing pressure ulcers.

According to still further features in the described preferred embodiments the configuration is any combination of a flat configuration, a flexible configuration and a thin configuration.

According to still further features in the described preferred embodiments the arrangement of sensors has a substantially uniform distribution of sensors.

According to still further features in the described preferred embodiments the arrangement of sensors has regions of higher sensor density and regions of lower sensor density.

According to still further features in the described preferred embodiments the regions of the higher sensor density correspond to body parts which are more prone at developing the ulcers.

According to still further features in the described preferred embodiments the body parts are selected from the group consisting of body parts most frequently in contact with the supporting-surface when the subject is lying on the back.

According to still further features in the described preferred embodiments the body parts are selected from the group consisting of the back of the head, the shoulders and upper back, the buttocks and upper thighs and the ankles and lower leg.

According to still further features in the described preferred embodiments each of the sensors comprise a pressure sensor.

According to still further features in the described preferred embodiments each of the sensors comprise an interfacial stress sensor.

According to still further features in the described preferred embodiments the interfacial stress sensor is selected from the group consisting of an interfacial contact stress sensor and an interfacial shear stress sensor.

According to still further features in the described preferred embodiments each of the sensors comprise a temperature sensor.

According to still further features in the described preferred embodiments each of the sensors comprise a humidity sensor.

According to still further features in the described preferred embodiments each of the sensors comprise at least two sensors selected from the group consisting of an interfacial stress sensor, a pressure sensor, a temperature sensor and a humidity sensor.

According to still further features in the described preferred embodiments each of the sensors comprise a pressure sensor and at least one additional sensor selected from the group consisting of a temperature sensor and a humidity sensor.

According to still further features in the described preferred embodiments each of the sensors comprise an interfacial stress sensor and at least one additional sensor selected from the group consisting of a pressure sensor, a temperature sensor and a humidity sensor.

According to still further features in the described preferred embodiments a portion of the at least one communication channel is wireless.

The present invention successfully addresses the shortcomings of the presently known configurations by providing a method and system for determining a risk of ulcer onset, which employs a procedure of evaluating internal tissue stresses based on skin surface loading.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of a portion of a skin (epidermis and dermis), a portion of a subcotaneous tissue underneath the skin and a portion of a muscle tissue underneath the subcotaneous tissue;

FIG. 2 is a schematic illustration of a system for determining a risk of pressure ulcer onset on a subject being in contact with a supporting-surface, according to the present invention;

FIG. 3 is a schematic illustration of a risk parameter calculator for calculating a risk parameter characterizing the risk of the ulcer onset, according to the present invention;

FIG. 4 is a flowchart of a method of determining a risk of pressure ulcer onset on a subject, according to the present invention;

FIG. 5 is a flowchart of a method of preventing ulcers to be formed on a subject, according to the present invention;

FIG. 6 is a schematic illustration of a device for determining contact parameters of a subject being in contact with a supporting-surface, according to the present invention;

FIG. 7a shows a prototype system, designed and constructed for determining a risk of pressure ulcer onset, according to the present invention;

FIG. 7b is a block diagram of the components of the prototype system shown in FIG. 7a, according to the present invention;

FIG. 8 is a matrix of 20 flexible contact compressive stress sensors used for the construction of the prototype system, according to the present invention;

FIG. 9 is a matrix of 8 skin temperature sensors used for the construction of the prototype system, according to the present invention;

FIG. 10 is a flowchart describing the operation of the prototype system, according to the present invention;

FIG. 11 shows a first screen of the prototype system display device which is to be used in the laboratory, according to the present invention;

FIG. 12 shows a second screen of the prototype system display device which is to be used, e.g., in hospital, according to the present invention;

FIG. 13 is a graph showing the pressure and the pressure dose in units of KPa as a function of time for a 10 Hz square signal;

FIG. 14 is a graph showing the pressure and the pressure dose in units of KPa as a function of time for a 10 Hz triangle signal;

FIG. 15 is a graph showing the pressure and the pressure dose in units of KPa as a function of time for a 10 Hz sine signal;

FIG. 16 is a graph showing the pressure and the pressure dose in units of KPa as a function of time for a 20 Hz sine signal;

FIG. 17a is a graph showing applied random contact compressive stress in units of KPa;

FIG. 17b is a graph showing calculated internal stress from the applied random contact compressive stress of FIG. 17a, in units of KPa;

FIG. 18 is a graph showing the pressure and the pressure dose in units of KPa as a function of time for the calculated internal stresses of FIG. 17b;

FIG. 19 is a graph showing applied random temperature pulses and the calculated temperature dose;

FIG. 20 is a graph showing calculated internal stress and the calculated pressure dose in units of KPa, for two applied pulse-sequences of a temperature pulse followed by a high-pressure pulse;

FIG. 21 is a graph showing the temperature and the calculated temperature dose in units of ° C., of the two applied pulse-sequences;

FIG. 22 is a graph showing the pressure dose index, temperature index and risk parameter as a function of time, for the two applied pulse-sequences;

FIG. 23 is a graph showing the internal stress and the pressure dose as calculated from contact compressive stress caused by a test subject, simultaneously measured with an applied temperature pulse, in units of KPa;

FIG. 24 is a graph showing temperature pulse applied simultaneously with a contact compressive stress caused by a test subject, and the calculated temperature dose in units of ° C.;

FIG. 25 is a graph showing the pressure dose index, temperature index and risk parameter as a function of time, for the internal stress and the temperature of FIGS. 23 and 24;

FIG. 26a shows a 3D model of a 5 mm-thick cross-section around the sacrum, according to the present invention;

FIG. 26b shows meshing of the model of FIG. 26a for finite element analysis;

FIGS. 27a-b show distribution of von Mises stresses during recumbency within the pelvis, according to the present invention;

FIG. 28a is a graph showing contact compressive stress under contact area of the 3D model with the supporting surface;

FIG. 28b is a graph showing the calculated internal von Mises stresses along the linear course S shown in FIG. 27b, resulting from the contact compressive stress of FIG. 28a;

FIG. 29a is an image of a special apparatus designed for applying calibrated constant compression for experimental use;

FIG. 29b shows a gracillis muscle positioned on a coarse sand-paper for experimental use;

FIGS. 30a-b show results of histological analysis of uninjured (a) and injured (b) gracillis muscles;

FIGS. 31a-b show an experimental setup used for ex vivo measurements of mechanical properties of excised muscles;

FIG. 32 show stress-strain curves obtained from measurements performed on excised gracillis muscles, using the experimental setup;

FIGS. 33a-c show tangent tensile moduli at strain percentage of 2.5% (a), 5% (b) and 7.5% (c);

FIGS. 34a-c show strain energy densities at strain percentage of 2.5% (a), 5% (b) and 7.5% (c);

FIGS. 35a-d show 3D models of 5 mm-thick anatomical slices of mattress-supported regions of the shoulders (a), heels (b), pelvis-sacrum (c) and head (d), according to the present invention; and

FIGS. 36a-c show distribution of von Mises internal stresses during recumbency within the shoulders, according to the present invention.

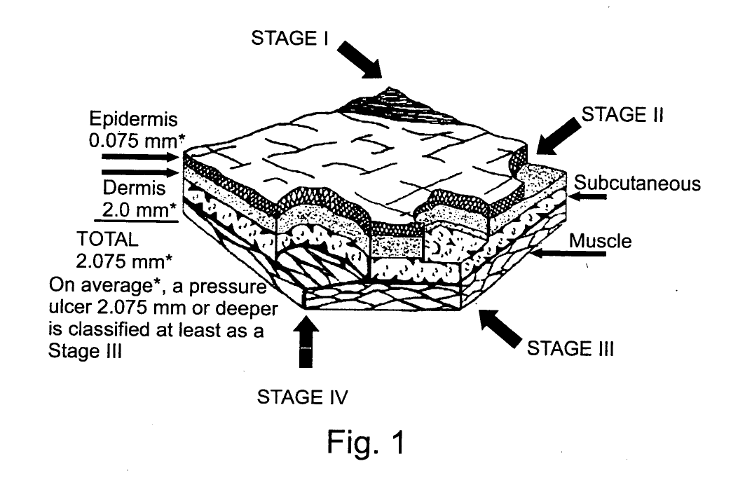

### DESCRIPTION OF THE PREFERRED EMBODIMENTS

The present invention is of a method and a system for determining a risk of pressure ulcer onset by measuring parameters such as the load on and the temperature of an organ over time. Specifically, the present invention can be used to alert prior to a formation of ulcers on a subject, so that the subject is moved, repositioned or otherwise treated, e.g., by nursing or hospital personnel or by an automatic mechanism. The present invention is further of a device for determining contact parameters of the subject for the purpose of determining and preventing onset of ulcers. The present invention is still further of a method of characterizing a risk of pressure ulcer onset.

The principles and operation of a method and a system for determining a risk of pressure ulcer onset according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

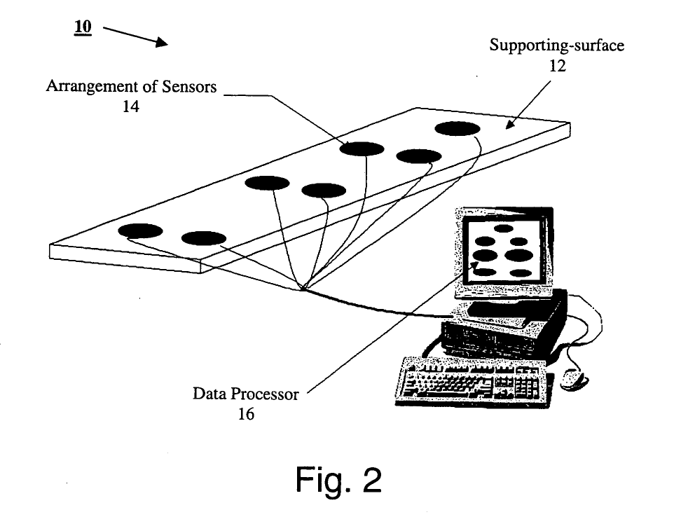

Referring now to the drawings, FIG. 2 illustrates a system 10 for determining a risk of pressure ulcer onset on a subject (not shown) being in contact with a supporting-surface 12. System 10 comprises an arrangement of sensors 14, located at predetermined locations between supporting-surface 12 and the subject, and a data processor 16 for receiving (e.g., by an A/D card) and processing data sensed by sensors 14.

Sensors 14 and data processor 16 are designed and programmed for determining the risk of pressure ulcer onset on the subject as further detailed hereinbelow. According to a preferred embodiment of the present invention sensors 14 and data processor 16 are also designed and programmed to alert (e.g., an audio-alert or a visual-alert) prior to a formation, on the subject, of ulcers adjacent to at least one sensor 14.

According to a preferred embodiment of the present invention sensors 14 may be integrated within or on supporting-surface 12, which may be, for example, a bed, a mattress, a chair, a wheelchair, an armchair, an operating table, a surface of a prosthesis being in contact with a residual limb (e.g., an inner surface of a leg or arm prosthesis) and the like. Sensors 14 may also be integrated within or on a clothing of the subject, either solely or in combination with other sensors (e.g., sensors which are integrated within or on supporting-surface 12). As system 10 is primarily directed at determining risk of pressure ulcers for the purpose of preventing such ulcers from occurring, each of sensors 14 is preferably of a flat configuration and designed not to substantially influence the risk of the subject at developing pressure ulcers.

The distribution of sensors 14 is not limited and may be either a uniform or non-uniform distribution. For example, the arrangement of sensors may have regions of higher sensor density and regions of lower sensor density, where the regions of higher sensor density correspond to body parts which are more prone at developing ulcers. Such body parts include, but are not limited to, body parts which are most frequently in contact with the supporting-surface when the subject is lying on the back, e.g., the back of the head, the shoulders and upper back, the buttocks and upper thighs, the ankles and lower leg, and the like.

As is stated in the Background section above, the time-frame for the formation of pressure ulcers depends on various factors. Thus, according to a preferred embodiment of the present invention the arrangement of sensors 14 may comprise any device capable of providing physical information concerning the contact between the subject and supporting-surface 12, e.g., any combination of interfacial stress sensors, temperature sensors, humidity sensors and the like. Preferably, at least one sensor is an interfacial stress sensor such as, but not limited to, a pressure sensor. In addition, integrated sensors for simultaneously sensing more than one measurable quantity (e.g., interfacial stress and temperature or temperature and humidity) may also be used.

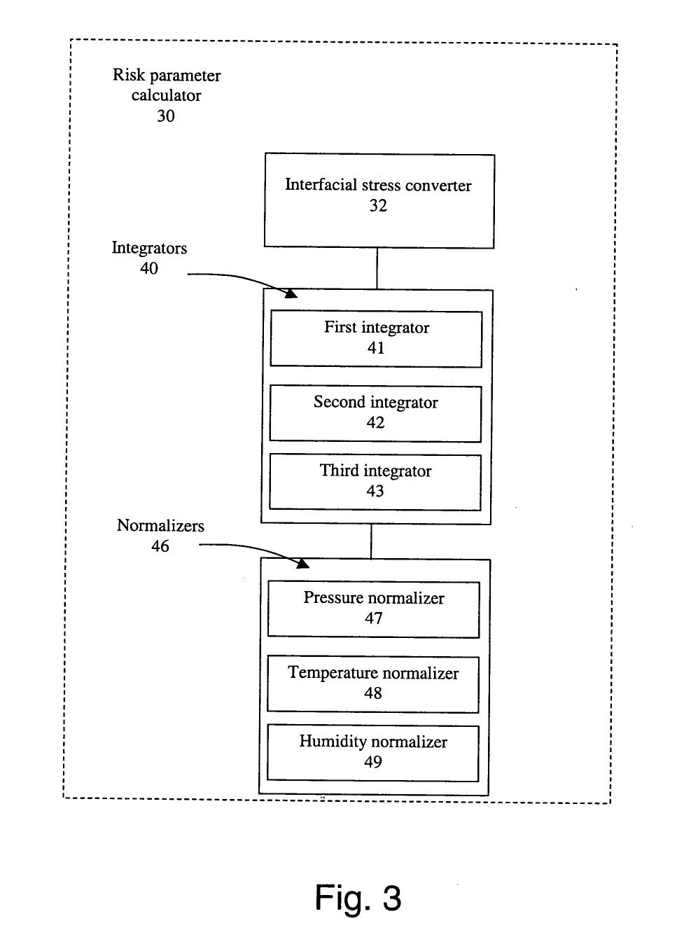

Reference is now made to FIG. 3, illustrating a risk parameter calculator 30 which, according to a preferred embodiment of the present invention, is used by data processor 16 for calculating a risk parameter, a, characterizing the risk of the ulcer onset. As further detailed hereinunder, risk parameter calculator 30 preferably calculates one or more dose indices for obtaining α. More preferably, risk parameter calculator 30 calculates at least two dose indices, e.g., a pressure dose index, PDI, a temperature dose index, TDI and a humidity dose index, HDI, so that α is a combination of these dose indices.

While conceiving the present invention, it has been unexpectedly uncovered, that that focal internal stresses in muscle tissue enveloping bony prominences is considerably higher than surface contact stresses. Therefore, severe pressure ulcers are more likely to be initiated within the body and only subsequently to progress outwards. Unlike prior art systems and methods, where the risk of pressure ulcers formation is determined solely based on interfacial pressures, the present invention successfully addresses the problem of calculating stresses in deep tissues so as to determine the pressure ulcers onset once initiated therein.

Hence, according to a preferred embodiment of the present invention, risk parameter calculator 30 comprises an interfacial stress converter 32, for converting interfacial stresses, as transmitted from sensors 14, into internal stresses. This preferably done by at least one conversion function, which may parameterized, for example, as a polynomial conversion function, an exponential conversion function, a rational conversion function, a power conversion function or any combination thereof. The conversion function may also be a look-up table, a set of look-up tables an interpolation function between one or more look up-tables or any other mathematical form of curve fitting.

Depending on the parameterization, the parameters of the conversion functions may be extracted from the literature, measured experimentally and/or calculated by an appropriate numerical model of the relevant anatomy. For example, the geometry of a particular part of the body (e.g., the pelvis, the head, the scapula, the sacrum, the buttocks, the pelvis, the heels etc.), may be constructed, based on real images (e.g., Ultrasound, MRI and/or CT or anthropometrical data measured or retrieved from the literature) and using a suitable computer software. Then, the constructed geometry may be used for solving an appropriate set of equations which correspond to skeletal, muscular, and other forces or internal pressures (e.g., abdominal) acting within the particular part of the body to obtain an internal stress distribution within tissues and organs. The set of equation may be solved using any numerical method for mechanical stress analysis such as, but not limited to, a method of finite-element solution. The obtained internal stress distribution is used for selecting the parameters of the conversion functions or for constructing the look-up tables. A detailed example of a model of the pelvis provided in the Examples section that follows.

The conversion functions (or the parameters thereof) may also depend on properties which vary from one individual to another, such as, but not limited to, the subject's weight and body structure, organ geometry, age, nutritional status, general health condition, presence of a disease affecting tissue mechanical properties, medications administered to the subject and the like. In addition, the conversion functions (or the parameters) may depend on one or more mechanical properties of the tissues (e.g., elastic and/or viscoelastic stress-strain relations and the coefficients of constitutive functions describing these relations). Mechanical properties which may affect the conversion functions include, but are not limited to, Young modulus, Poisson's ratio, shear modulus, bulk modulus, Lamé coefficients, tangent elastic modulus, one or more hyper-elastic material model invariants and one or more coefficients which characterize a certain phenomenological function describing experimental stress-strain data. Alternatively or in addition, the conversion functions (or the parameters) may depend on mechanical interaction between the body and supports, such as, but not limited to, geometrical and mechanical characteristics of the supports, the body-support friction characteristics, etc.

Preferably, the conversion functions depends on dimensionless mechanical properties, which may be obtained, for example, by dividing each mechanical property of the subject by a respective reference mechanical property (e.g., of a normal young subject). Also, varying the conversion functions for different psychosocial states is not excluded from the scope of the present invention.

Mathematically, the conversion of interfacial stresses into internal stresses is written in the form:
σᵢₙₜ=F(σ_{surface}, {Vᵢ}) (EQ. 1)
where, F is the conversion function, σᵢₙₜ is the internal stress, σ_{surface} is the interfacial stress and {Vᵢ} i=1, . . . , N, is a set of variables or parameters of which F depends, as detailed above. For example, in one embodiment F is a polynomial function, Pol, and the set {Vᵢ} comprises the subject's weight, W, and the ratio between the Young modulus of the subject to the Young modulus of a normal subject, which ratio is known [Gefen et al., Med. Biol. Eng. Comput. 1999, 37, 625-631] as tissue-stiffness-ratio and denoted herein by K. Hence, in this embodiment, Equation 1 is reduced to the form:
σᵢₙₜ=Pol(σ_{surface}, W, κ). (EQ. 2)

According to a preferred embodiment of the present invention the calculation of the dose indices is by integration, thus, as further detailed below, risk parameter calculator 30 preferably comprises at least one integrator 40 for performing integration thereby to obtain the dose indices.

According to a preferred embodiment of the present invention, integrators 40 comprise a first integrator 41, for integrating the internal stress over a first predetermined period, thereby to provide the PDI. The data sensed by sensors 14 in general, and the interfacial stresses in particular, are preferably transmitted regularly (either continuously or in predetermined time intervals). Thus, the integration of the internal stress is preferably an integration over a time-dependent quantity, σᵢₙₜ(τ), where τ is a time variable. The PDI is therefore calculated according to the following equation:
PDI=∫σᵢₙₜ(τ)dτ. (EQ. 3)

The first predetermined period, which is the elapsed time between the lower and the upper limits of integration of Equation 3, is preferably proportional to the time scale of the pressure ulcers to be formed and it may vary from one subject to another.

Integrators 40 may further comprise a second integrator 42, for integrating the temperature transmitted from sensors 14 over a second predetermined period, thereby to obtain the TDI. Similarly to the PDI, the TDI is preferably calculated using Equation 4 below, where the second predetermined period is the elapsed time between the lower and the upper limits of integration
TDI=∫T(τ)dτ. (EQ. 4)

According to a preferred embodiment of the present invention integrators 40 may further comprise a third integrator 43, for integrating the humidity transmitted from sensors 14 over a third predetermined period, thereby to obtain the HDI. Similarly to the PDI and the TDI above, the HDI is preferably calculated using Equation 5 below, where the third predetermined period is the elapsed time between the lower and the upper limits of integration
HDI=∫H(τ)dτ. (EQ. 5)

The first, second and third predetermined periods are not limited and may vary according to the subject's characteristics, support characteristics and/or other parameters of the mechanical interaction. A typical first, second and third predetermined periods is about one hour.

As used herein, the term about refers to ±10%.

Risk parameter calculator 30 may further comprise one or more normalizers 46 for normalizing a respective dose index. More specifically, normalizers 46 comprise a pressure normalizer 47 for normalizing the PDI, using a reference pressure dose. Preferably, the reference pressure dose is selected so that if the PDI exceeds a unity then the subject is substantially at risk of the ulcer onset at a respective location. This may be done by selecting the reference pressure dose to be the maximal pressure dose for which no damage is caused to living muscular tissue under constant compression for the first predetermined period. In addition, normalizers 46 preferably comprise a temperature normalizer 48 for normalizing the TDI, using a reference temperature dose, which may be, for example, proportional to the second predetermined period. Still in addition, normalizers 46 preferably comprise a humidity normalizer 49 for normalizing the HDI, using a reference humidity dose, which may be, for example, proportional to the third predetermined period.

Once the dose indices are calculated, a is obtained. Specifically, in embodiments in which only one dose index PDI is used (e.g., the PDI):
α=PDI, (EQ. 6)
in the embodiment in which more that one dose index is used (e.g., the PDI and the TDI and/or the HDI), α is obtained by an appropriate combination of the dose indices. A simple way to obtain α is by multiplying the calculated dose indices,
α=PDI·TDI, (EQ. 7)
or
α=PDI·TDI·HDI. (EQ. 8)

However, other combinations of the dose indices may be used as well, for example, a convolution, a power-law combination, a linear combination, etc. In addition, α may also be obtained by a logic decision procedure, such as but not limited to, a procedure which selects the highest dose index and the like.

As stated, sensors 14 and data processor 16 are designed and programmed to alert prior to a formation, of at least one ulcer. The advantage of defining and using the risk parameter (e.g., using one Equations 6-8) is that it can serve as an alert trigger. In other words, the alert is generated if a is above a predetermined threshold. Another advantage of defining and using the risk parameter is that it may be used for displaying a risk status of the subject either globally or for each of the locations where sensors 14 are located. Hence, system 10 preferably comprises a display 38.

According to a preferred embodiment of the present invention system 10 may further comprise a communication channel for transmitting information from system 10 to a remote location, which may be, for example, a nursing control center in a medical or healthcare institution. The remote location may also be a physicians center (or place of residence) so that as to allow valuable information to be transmitted to a physician without delay. Hence, the communication channel is preferably connected to a telemetry apparatus or telemedicine apparatus.

According to a preferred embodiment of the present invention the risk status of the subject may be stored, in a retrievable and/or displayable format on memory media for future use.

According to a preferred embodiment of the present invention the memory media can be any memory media known to those skilled in the art, which is capable of storing the risk status either in a digital form or in an analog form. Preferably, but not exclusively, the memory media is removable so as to allow plugging the memory media into a host (e.g., a processing system), thereby allowing the host to store the risk status in it or to retrieve the risk status from it.

Examples for memory media which may be used include, but are not limited to, disk drives (e.g., magnetic, optical or semiconductor), CD-ROMs, floppy disks, flash cards, compact flash cards, miniature cards, solid state floppy disk cards, battery-backed SRAM cards and the like.

According to a preferred embodiment of the present invention, the risk status is stored in the memory media in a retrievable format so as to provide accessibility to the stored data. Preferably, information is retrieved from the memory media either automatically or manually. That is to say that some library of risk status may be constructed and searched thereafter by an appropriate set of search codes, or alternatively, a user may scan the entire library or a portion of it, so as to find a match for a query risk status. According to a preferred embodiment of the present invention the risk status is stored in the memory media in more than one form.

Hence, in one embodiment the risk status is stored as one or more images displaying, e.g., a graph of certain measured quantity over time or a map displaying locations which are more sensitive to pressure ulcers. In another embodiment, the risk status is stored in a textual format which facilitates using search codes.

It is appreciated that in all the above embodiments, risk status data are stored in the memory media in an appropriate displayable format, either graphically or textually. Many displayable formats are presently known, for example, TEXT, BITMAP™, DIF™, TIFF™, DIB™, PALETTE™, RIFF™, PDF™, DVI™ and the like. However it is to be understood that any other format that is presently known or will be developed during the life time of this patent, is within the scope of the present invention.

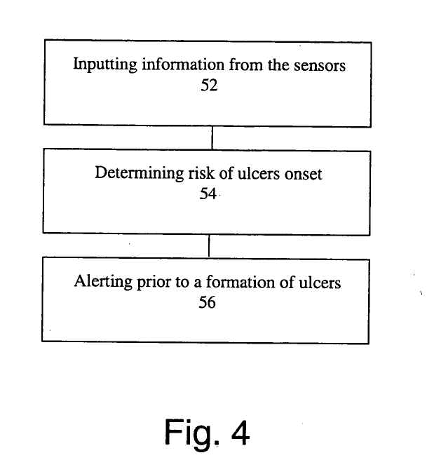

Reference is now made to FIG. 4, which is a flowchart of a method of determining a risk of pressure ulcer onset on a subject being in contact with a supporting-surface, according to another aspect of the present invention. The method comprises the following method steps which may be executed by an appropriate device or system, e.g., system 10.

Hence, Block 52 represents a first step in which information is inputted from an arrangement of sensors, located on predetermined locations between the supporting-surface and the subject. Block 54 represents a second step in which the inputted information is used for determining the risk of pressure ulcer onset on the subject. The risk of pressure ulcer onset may be determined by calculating the risk parameter, α, as further detailed hereinabove. According to a preferred embodiment of the present invention the method may further comprise a third step, represented by Block 56, in which an alert is produced prior to a formation of pressure ulcers.

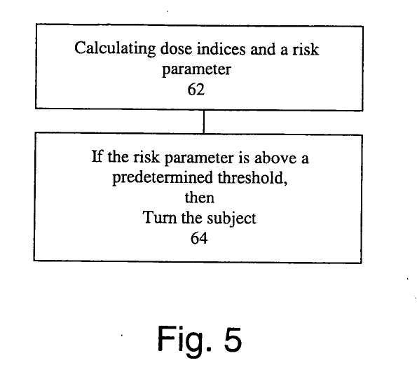

Reference is now made to FIG. 5, which is a flowchart of a method of preventing ulcers to be formed on a subject, according to still another aspect of the present invention. The method comprises the following method steps in which in a first step, represented in FIG. 5 by Block 62, a plurality of dose indices and a risk parameter, α, are calculated. The dose indices and a risk parameter are preferably calculated as detailed above with respect to the operation of system 10. In a second step, represented by Block 64, the position of the subject is changed (e.g., the subjects is turned on the side) if the risk parameter is above a predetermined threshold.

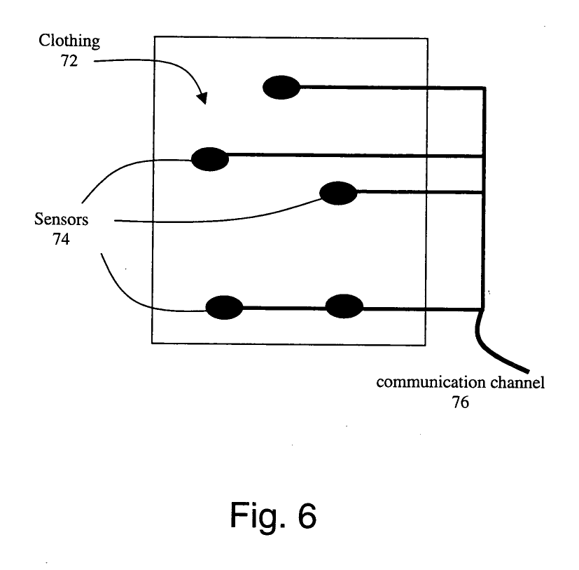

Reference is now made to FIG. 6, which is a schematic illustration of a device for determining contact parameters (e.g., stress, pressure, temperature or humidity) of a subject being in contact with a supporting-surface, according to still another aspect of the present invention. Hence, the device comprises clothing 72 and an arrangement of sensors 74 which are integrated within or on clothing 72. The device further comprises at least one communication channel 76 (e.g., wires or wireless transmitters) for transmitting information sensed by sensors 74. According to a preferred embodiment of the present invention, sensors 74 may be similar to sensors 14 of system 10.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples which, together with the above descriptions, illustrate the invention in a non limiting fashion.

### Example 1

### A Prototype System

A prototype of a system for determining a risk of pressure ulcer onset according to the above description was constructed.

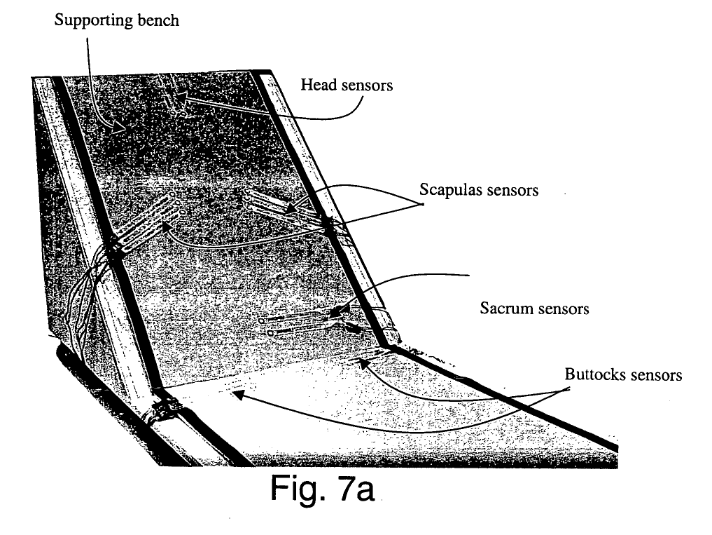

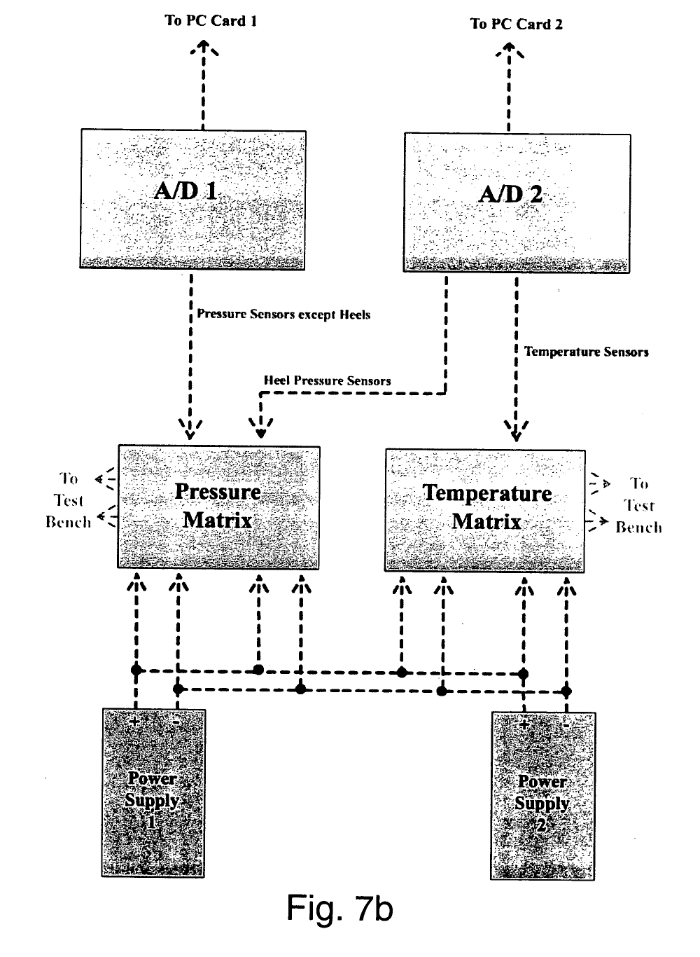

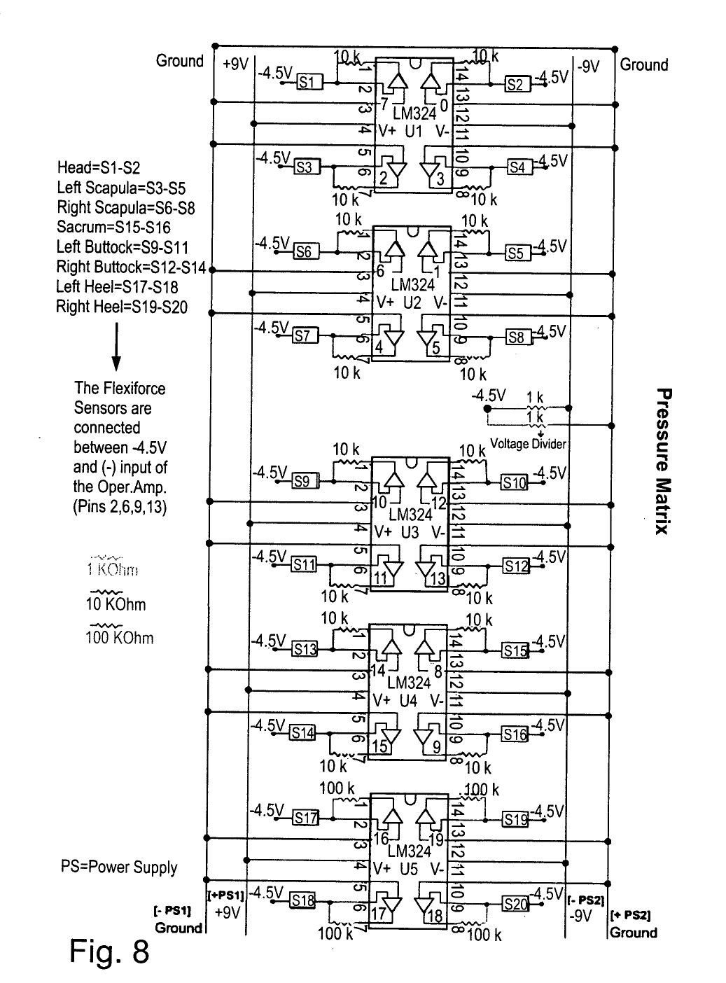

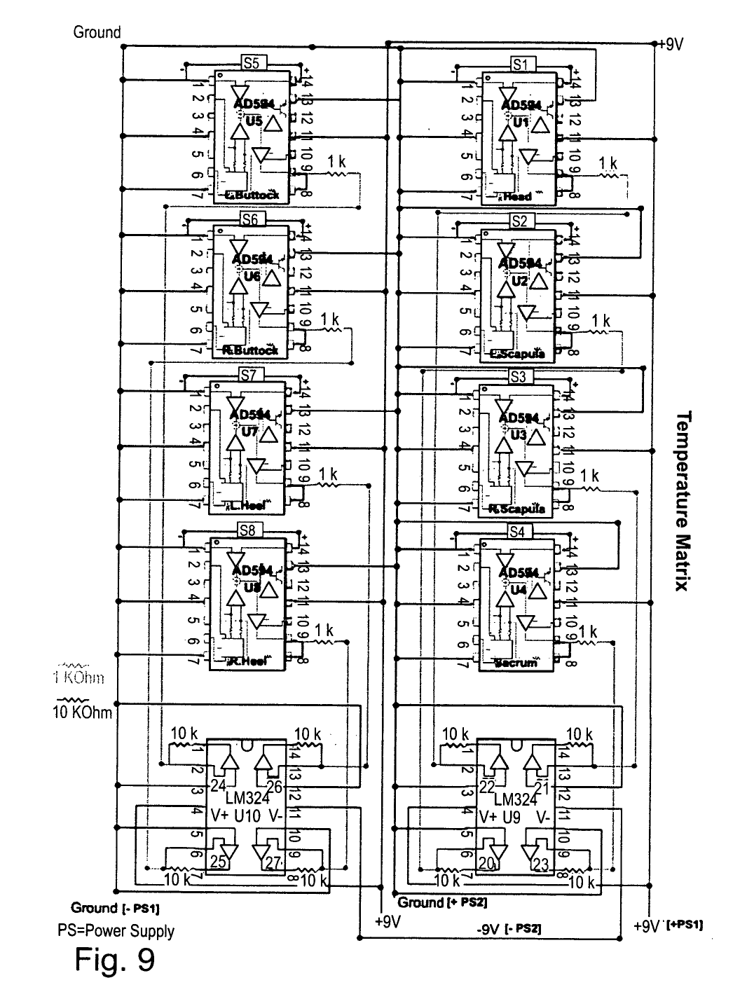

Reference is now made to FIG. 7a which is a photographed image of the prototype system, and to FIG. 7b which is a block diagram of the components of the prototype system. The prototype system includes:
(a) a supporting bench;
(b) a matrix of 20 flexible contact compressive stress sensors, of type Flexiforce, purchased from Tekscan Inc., the matrix is further detailed in FIG. 8;
(c) a matrix of 8 skin temperature sensors of type T Thermocouple, purchased from Tekscan Inc., the matrix is further detailed in FIG. 9; and
(d) a data processor having two 16-channeled A/D cards of type PCI-6023, purchased from National Instrument.

A complete summery of all the components used for the construction of the prototype system (sensors, connectors, cards, cables, resistors, etc.) is provided in Table 1.

TABLE 1

| | Quantity | Identification | Manufacturer |
|---|---|---|---|
| Contact compressive stress Sensor | 16 | FlexiForce | Tekscan Inc. |
| (with 3-pin Male Berg Connector) | | A101-1 | |
| | | 0-1 lb. (4.4 N) | |
| Contact compressive stress Sensor | 4 | FlexiForce | Tekscan Inc. |
| (with 3-pin Male Connector) | | A101-25 | |
| | | 0-25 lbs. | |
| | | (110 N) | |
| 3-pin Female Connector | 20 | #65801-003 | Tekscan Inc. |
| Thermocouple | 8 | Type T | Omega |
| (2 m Teflon cable with Male | | | |
| Miniature Connector) | | | |
| Female Miniature Connector | 8 | Type T | Omega |
| Thermocouple Amplifier | 8 | AD594 | Analog Devices |
| Operational Amplifier | 7 | LM324N | National |
| | | | Semiconductor |
| A/D Card | 2 | PCI-6023E | National Instruments |
| Conductor Ribbon Cable | 2 | R6868 | National Instruments |
| Connector Block | 2 | CB-68LP | National Instruments |
| DC Power Supply (0-30 V, 0-3 A) | 1 | P3030 | Advice |
| DC Power Supply (0-25 V, 0-6 A) | 1 | RGPS 18-6 | Gamatronic |
| Hook-Up Wire (3 m × 1.09 mm) | 8 | | |
| Hook-Up Wire (2 m × 1.09 mm) | 32 | | |
| Hook-Up Wire (1 m × 1.09 mm) | 28 | | |
| Hook-Up Wire (0.5-1 m × 3 mm) | 8 | | |
| Jumper Wire (0.5-5 cm × 1 mm) | 100 | | |
| Breadboard | 3 | | |
| Banana Connector | 8 | | |
| Wire Up Pin | 8 | | |
| 100 KΩ Resistor | 4 | | |
| 10 KΩ Resistor | 24 | | |
| 1 KΩ Resistor | 10 | | |
| Hospital Bed Model | 1 | | |
| Mattress | 1 | | |
| Velcro | 1 | | |
| Corrugated Tubing | 1 | | |
| Plug (Male & Female) | 5 | | |
| Protective Box | 4 | | |

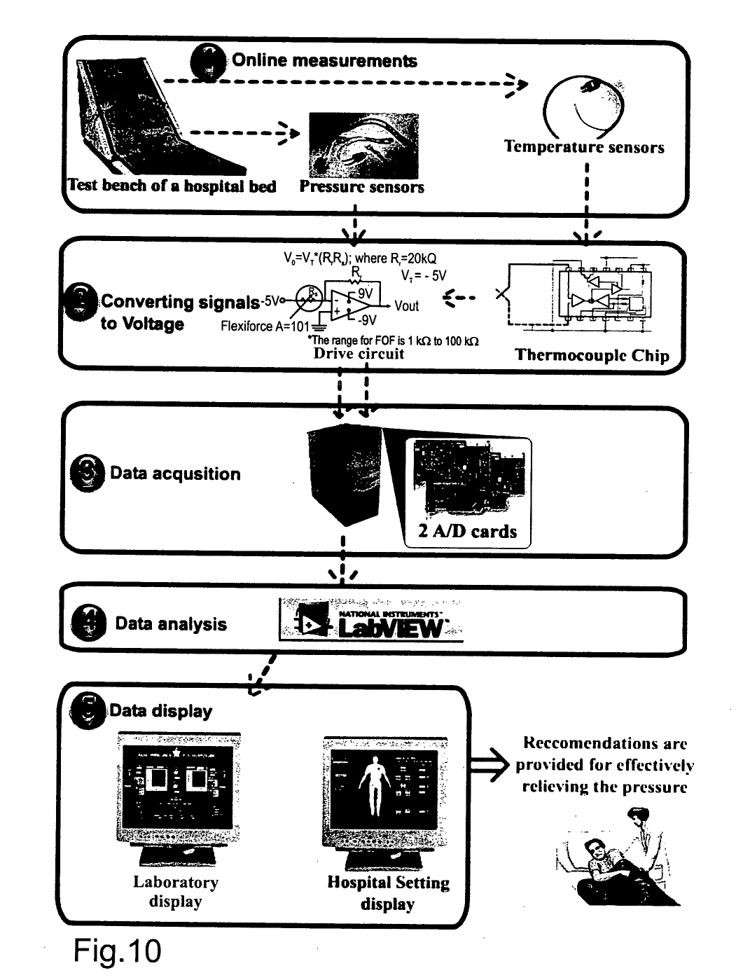

The operation of the prototype system is depicted in the flowchart of FIG. 10, where Block 1 represents online measurement of the pressure and the temperature using the sensors. The sensors were sampled simultaneously and continuously. Block 2 represents amplification of the signals transmitted from the sensors. The signals from the temperature sensors were amplified using a thermocouple amplifier of type AD594 purchased from National Semiconductor Ltd., and signals from the pressure sensors were amplified using an operational amplifier of type LM324N purchased from Analog Devices Ltd.

Block 3 represents data acquisition of the amplified signals. The sensors were sampled simultaneously and continuously by the A/D cards.

Block 4 represents the analysis of the data using a LabView 6i algorithm, as further detailed hereinbelow.

The temperature and pressure signals were directly sampled (sampling rate of 0.1 second) from the A/D cards to provide unprocessed voltage values. These values Were converted into pressure and temperature by linear calibration functions which were determined experimentally in a separate experiment. A pressure threshold of 0.4 KPa was imposed to minimize noise artifacts. Pressure values above the pressure threshold, were converted from external contact compressive stress to internal tissue stress using a conversion function derived from a biomechanical model which is further detailed in Example 4 below.

Pressure dose and temperature dose indices were calculated and normalized as detailed above using constant time periods of 1 hour. The pressure dose index was normalized by a reference pressure dose which was selected to be the maximal pressure dose that can be applied without causing local necrosis of the muscle. The reference temperature dose was selected to be the maximal temperature dose that the skin can bear without local sweat response. The calculation time was of the order of milliseconds, hence, no significant delays were caused through the acquisition, storage and processing of data.

The system was programmed to provide alerts according to the values the risk parameter, α. Three independent risk parameters were tested: α=PDI, α=TDI and α=PDI·TDI. In addition the system recommends a relief of pressure under specific areas in the subject's body.

Referring again to FIG. 10, Block 5 represents the output of the data analysis to a display device. Two screens were designed, a first screen to be used in the laboratory and a second screen to be used while the system is in operation, e.g., in hospital. When in operation, the display device shows information regarding the specific locations of risk.

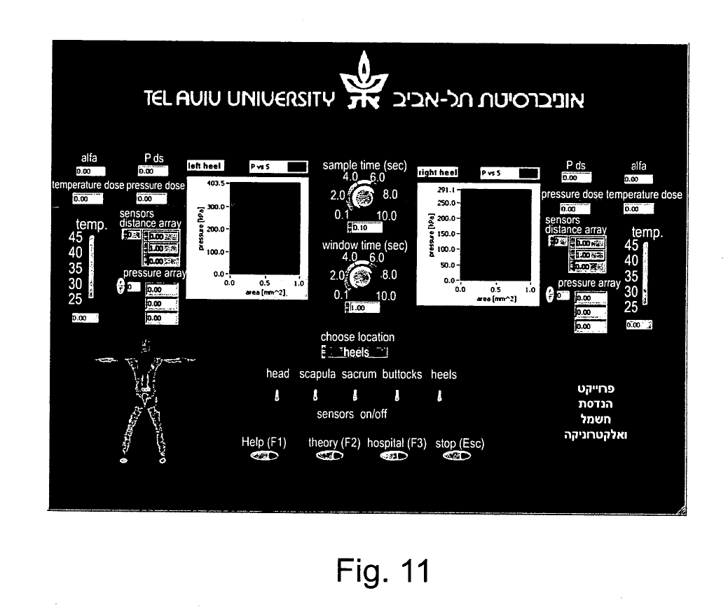

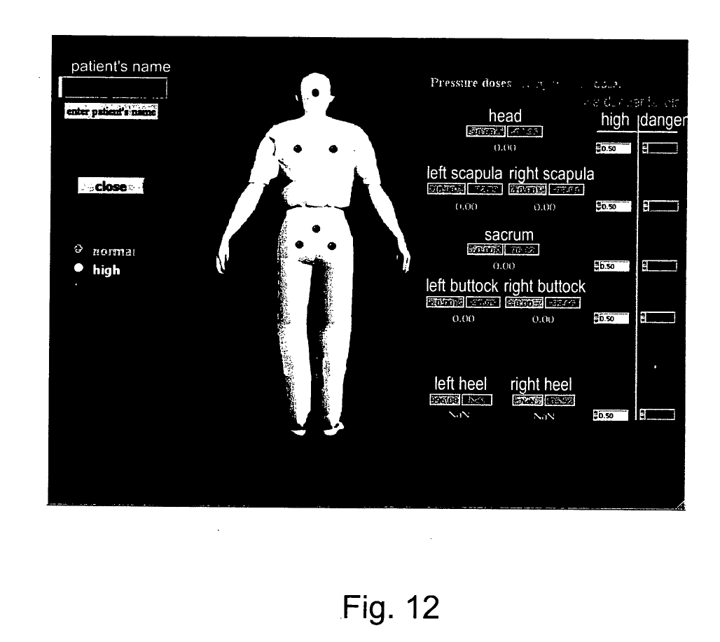

Reference is now made to FIG. 11, showing the first screen which is to be used in the laboratory. The screen provides detailed information of all the data of a specific, pre-selected, body part at a time (e.g., head, scapula, sacrum, buttocks or heels), together with a visual representation of the selected body part.

Reference is now made to FIG. 12, showing the second screen which is to be used in operation, e.g., in hospital. The screen provides the pressure dose, the temperature dose and the risk parameter simultaneously for all monitored body parts, which are, in the present example, the head, the left scapula, the right scapula, the sacrum, the left buttock, the right buttock, the left heel and the right heel. In addition, the risk level of pressure ulcer onset, according to the value of α, is displayed as a color coded mark on a respective location of a body image (a first color for danger, a second color for high risk and a third color for normal state).

### Example 2

### Simulations

Simulations of different pressure and temperature profiles were performed by producing synthetic voltage signals to simulate sensor outputs. Instruments used in this example and in Example 3 below, are listed in Table 2.

TABLE 2

| | Quantity | Identification | Manufacturer |
|---|---|---|---|
| Multi-meter | 1 | 73-3 | Fluke |
| Oscilloscope | 1 | TDS210 | Tektronix |
| Function Generator | 1 | CFG253 | Tektronix |
| Aluminum Pressure Weight | 15 | | |
| Mercury Thermometer | 1 | | |

Four different profiles were tested: a 10 Hz square signal, a 10 Hz triangle signal, a 10 Hz sine signal and a 20 Hz sine signal, all of which having amplitudes of 4-5 Volts. The simulations were executed using a sample time of 0.01 seconds and a time frame of 1 second.

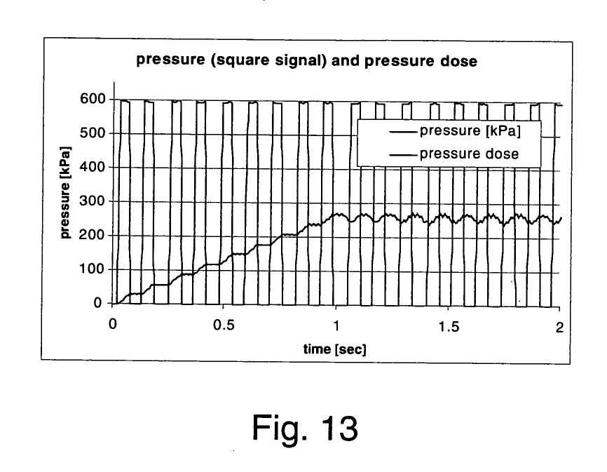

With reference to FIG. 13, a square signal of about 4.33 V (Duty Cycle=50%) was produced to simulate pressure readings. The voltage was converted to contact compressive stress of about 21.65 KPa (see Example 1) and then to internal stress of about 589.83 KPa. Since the pressure is accumulated gradually, initially (for 1 second, the selected time frame) the pressure dose rises with each pulse and stays constant when the pulse is off After the first second the maximum pressure dose value is 294.9 KPa, which is half the value of the internal stress. The pressure dose fluctuates near this value according to the pulses. FIG. 13 shows the pressure and the pressure dose in units of KPa as a function of time for the 10 Hz square signal.

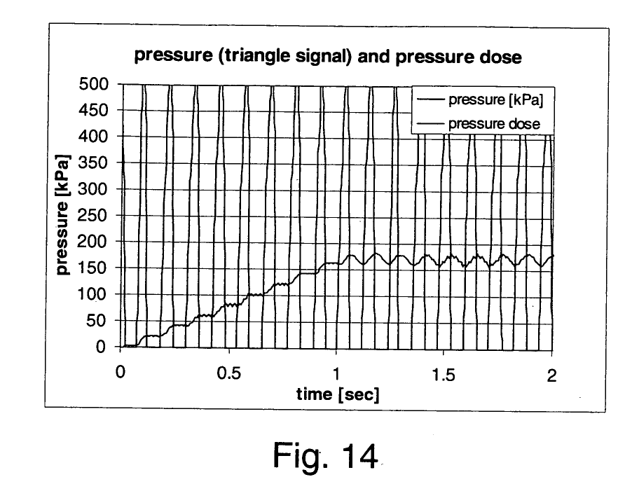

FIG. 14 shows the pressure and the pressure dose in units of KPa as a function of time for the 10 Hz triangle signal. As seen in FIG. 14, the pressure dose reaches a lower value than half the amplitude of the signal for a triangle signal.

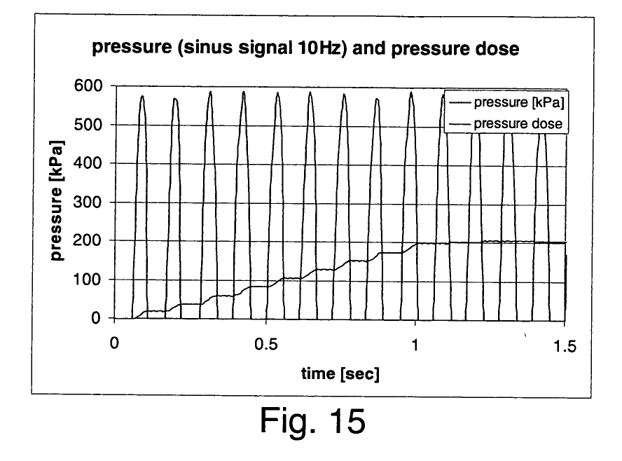

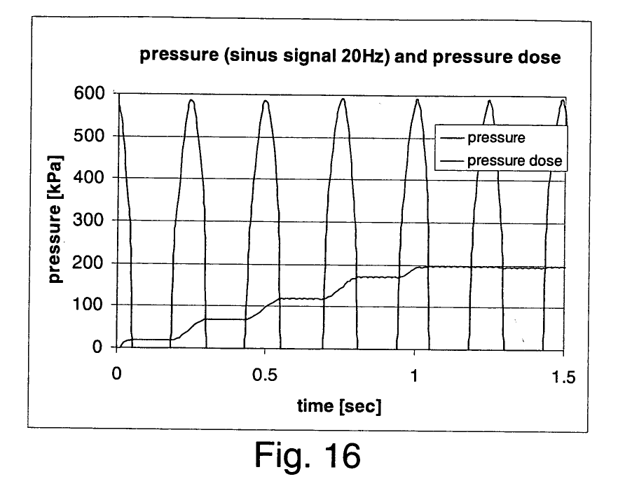

FIG. 15 and FIG. 16 show the pressure and the pressure dose in units of KPa as a function of time for the 10 Hz and 20 Hz sine signals, respectively. Both sine profiles yielded a 200 KPa pressure dose, where the buildup shape of the final pressure dose during the first second depends on the frequency of the input signal.

### Example 3

### Direct Contact Tests

The sensors were stimulated in the laboratory in four different experiments. As in Example 2, the sample time was 0.1 sec and the time frame was 1 sec.

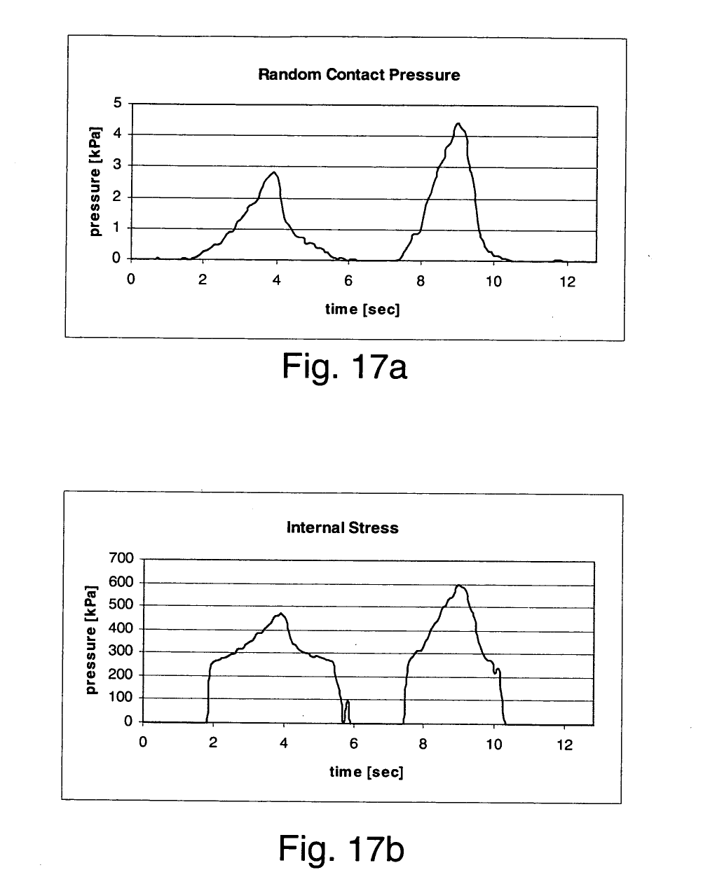

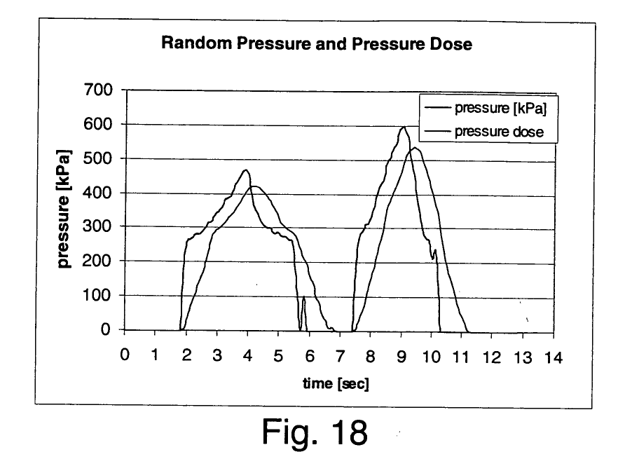

With reference to FIG. 17a, FIG. 17b and FIG. 18, in a first experiment two random pressure pulses were applied to the pressure sensors by a direct contact. The contact compressive stress, from 0 KPa to about 4.5 KPa, was converted to internal stress, ranging from 0 KPa to about 600 KPa. As discussed in Example 1, pressure under 0.4 KPa was filtered out, i.e., considered as internal stress of 0 KPa. FIG. 17a shows the applied random contact compressive stress in units of KPa and FIG. 17b shows the calculated internal stress in units of KPa. FIG. 18 shows the pressure and the pressure dose in units of KPa as a function of time for the calculated internal stresses of FIG. 17b. It is shown that the pressure dose trace follows the applied pressure signal over the duration of the two random pulses.

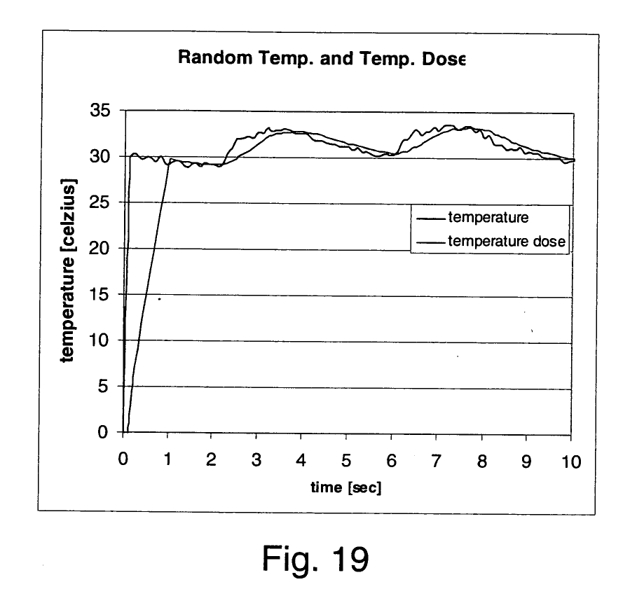

With reference to FIG. 19, in a second experiment two random temperature pulses were applied to the temperature sensors by a direct contact. FIG. 19 shows the applied random temperature pulses and the calculated temperature dose. It is shown that the temperature dose trace follows the measured temperature over the duration of the two random temperature pulses.

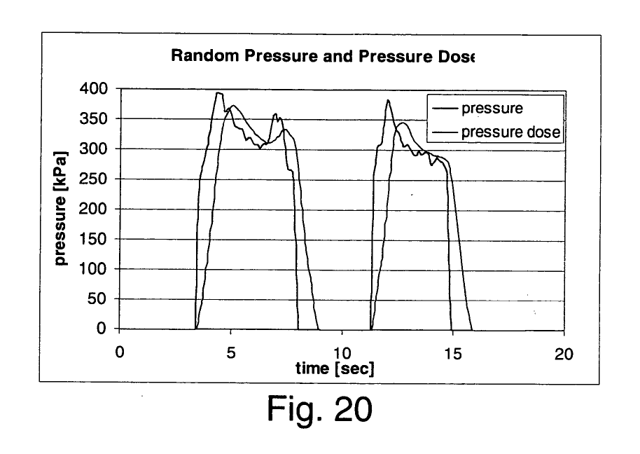

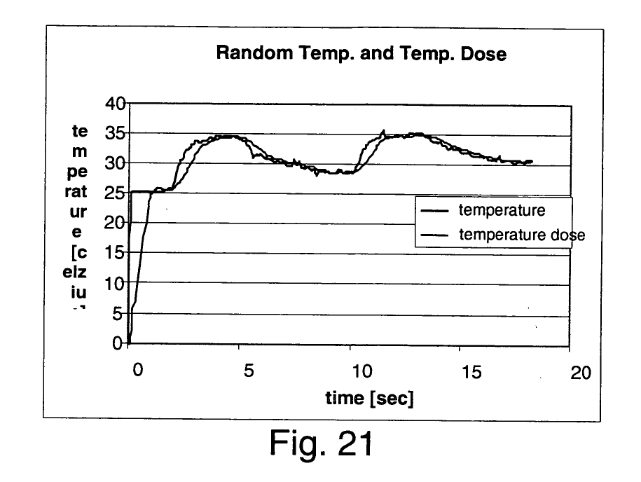

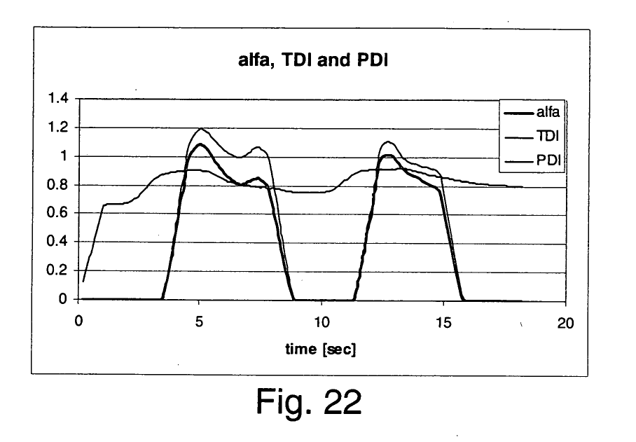

With reference to FIG. 20, FIG. 21 and FIG. 22, in a third experiment two pulse-sequences of a temperature pulse followed by a high-pressure pulse were applied. FIG. 20 shows the calculated internal stress and the calculated pressure dose in units of KPa, and FIG. 21 shows the applied temperature pulse and the calculated temperature dose in units of ° C. The internal stress and the temperature were normalized by a reference temperature dose and a reference pressure dose, as further detailed above, to produce the pressure dose and the temperature indices, PDI and TDI, respectively. The risk parameter was then calculated as a product of the two indices (see Equation 7). FIG. 22 shows the (dimensionless) pressure dose index, temperature index and risk parameter as a function of time. As can be seen, the risk parameter is reduced by the temperature index however does exceeds the value of unity for both sequences.

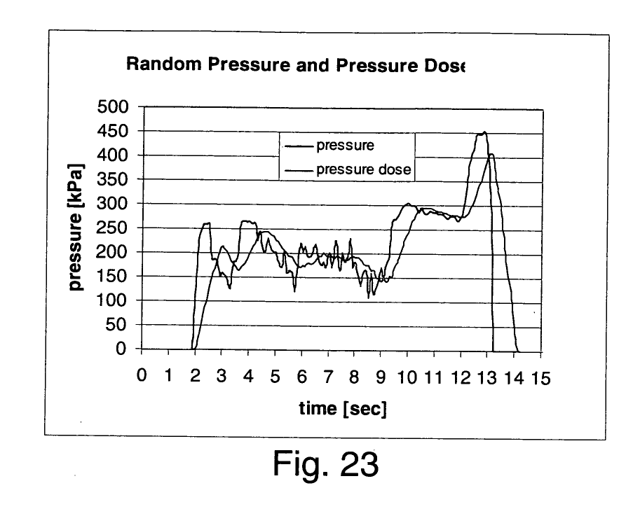

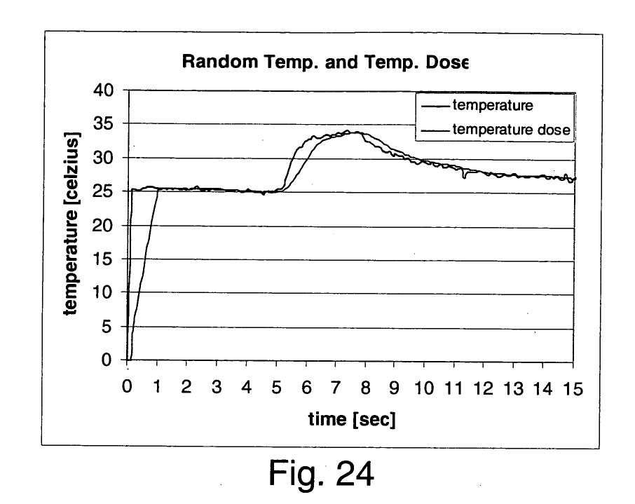

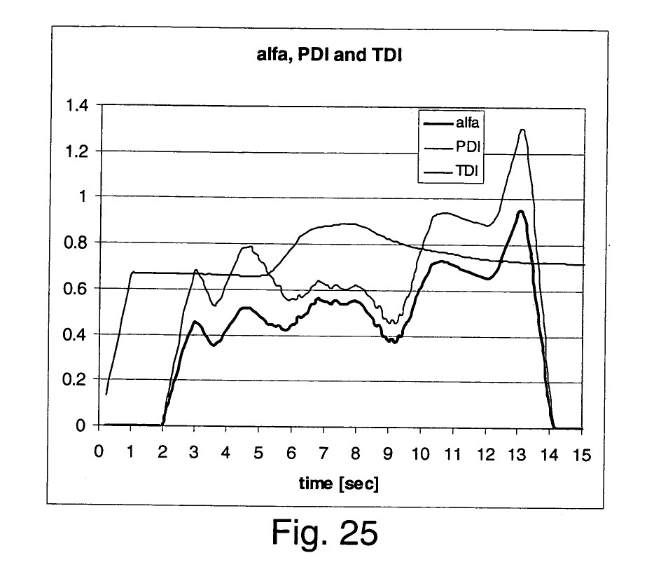

With reference to FIG. 23, FIG. 24 and FIG. 25, in a fourth experiment one temperature pulse was applied simultaneously with a measurement of contact compressive stress of the left scapula of a test subject. FIG. 23 shows the internal stress and the pressure dose as calculated from the measured pressure in units of KPa, and FIG. 24 shows the applied temperature pulse and the calculated temperature dose in units of ° C. FIG. 25 the (dimensionless) pressure dose index, temperature index and risk parameter as a function of time. As can be seen, the risk parameter did not exceeds the value of unity in this experiment.

### Example 4

### A Model for Calculating Internal Stresses

A realistic, anatomically accurate three-dimensional (3D) model of the pelvis, was developed, by reconstructing a 3D anatomy of a 5 mm-thick cross-section around the sacrum which was obtained from the Visible Human (male) digital database produced by the US Natioal Library of Medicine (NLM) [http://www.nlm.nih.gov/research/visible/visible_human.html].

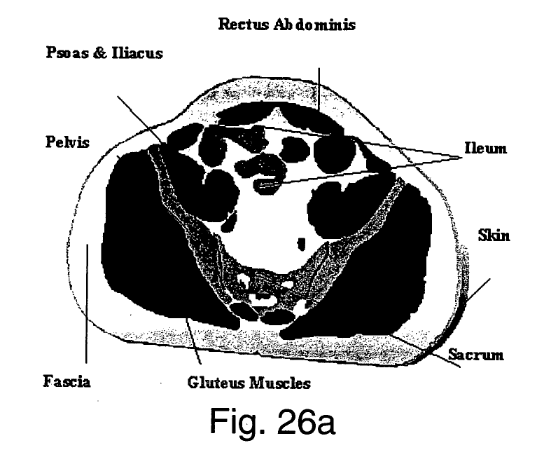

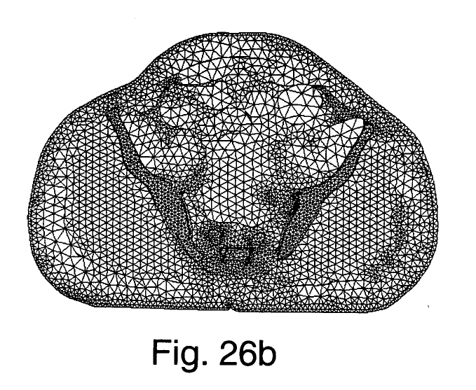

FIGS. 26a-b show the 3D slice geometry which was obtained in the following manner. The bones, cartilage, muscles, colon, ileum, major blood vessels, fascia and skin, shown in FIG. 26a, Were segmented and their contours were transferred to a solid modeling software package SolidWorks® 2001. The slice was then represented as shown in FIG. 26b by solid volumes which were obtained by the solid modeling software package.

For the purpose of structural analysis under loading, tissues were assumed to be homogenous, isotropic and non-linear elastic materials. For the bones, the Young modulus and the Poisson ratio were selected to be 7300 N/mm² and as 0.3, respectively [Nakamura, S. et al., 1981, “An Analysis of Soft Tissue Loading in the Foot: A Preliminary Report”, Bull. Prosth. Res., 18: 27-34]. For the cartilage, Young modulus and Poisson ratio were selected to be 10 N/mm² and 0.1, respectively [Liu, G. T. et al., 1997, “Human Articular Cartilage Biomechanics of the Second Metatarsal Intermediate Cuneiform Joint”, J. Foot Ankle Surg., 36: 367-374; Athanasiou, K. A. et al., 1998, “Biomechanical Topography of Human Articular Cartilage in the First Metatarsophalangeal Joint”, Clin. Orthop., 348: 269-281]. The Young modulus of colon and ileum was determined as 47 N/mm² and their Poisson ratio as 0.5 [Todd B. A., Thacker, J. G. (1994) “Three-dimensional computer model of the human buttocks, in vivo”, J Rehabil Res Dev 31: 111-9]. The major blood vessels were modeled as having Young modulus of 0.3 N/mM² and Poisson ratio of 0.4 [How, T. V. (1992): Mechanical properties of arteries and arterial grafts, in Hastings, G. W. (Ed.): Cardiovascular Biomaterials (London, Springer-Verlag), pp. 1-35]. The skin, muscles and fat were modeled as being non-linear elastic with their stress-strain relations based on experimental data [e.g., Gefen, A., Megido-Ravid, M., Itzchak, Y. (2001) “In vivo biomechanical behavior of the human heel pad during the stance phase of gait”, J Biomech 34: 1661-5].

Once the 3D slice geometry was obtained and the materials properties were determined, the geometry was transferred to a finite element solver, NASTRAN® 2001, for structural analysis under body loading in a recumbent posture.

Predictions of internal stress distributions were validated experimentally by comparing simulated to measured contact compressive stresses, which were acquired by means of polyester pressure sensitive films purchased from Sensor Products Ltd. Simulations of representative stress states in vulnerable populations (elderly and diabetics) were developed by increasing the stiffness of the skin and fascia to 6 times their normal values, in order to mimic the results of aging or disease. For example it has been found that for diabetic patients with abnormally stiff tissue properties as specified in an article by Gefen et al., entitled “Integration of plantar soft tissue stiffness measurements' in routine MRI of the diabetic foot”, published in Clin. Biomech., 2001, 16: 921-925, the maximal stresses may rise by 33% at the surface and by 22% internally, around the sacrum.

For each solid volume, the distribution of von Mises stresses were calculated. The von Mises equivalent stresses (σ_{v.M.}) weight the effect of all principal stresses (σ₁, σ₂, σ₃) according to the equation:

$${σ}_{v . M .} = {\{ \frac{1}{2} [ {( {σ}_{1} - {σ}_{2} )}^{2} + {( {σ}_{2} - {σ}_{3} )}^{2} + {( {σ}_{3} - {σ}_{1} )}^{2} ] \}}^{\frac{1}{2}} \qquad ( EQ . 9 )$$

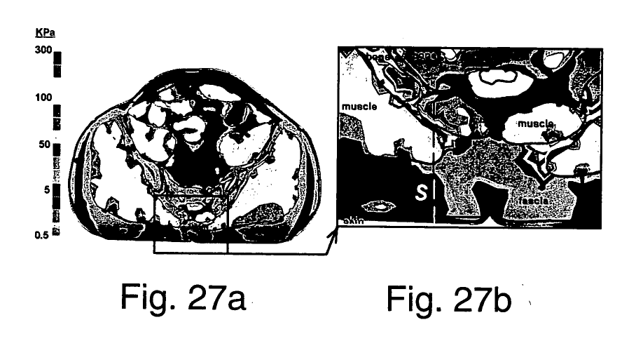

Reference is now made to FIG. 27a and FIG. 27b which show resulted distribution of von Mises stresses during recumbency within the pelvis. FIG. 27a shows the entire slice while FIG. 27b shows a magnified region of interest under the bony prominences. Also shown in FIG. 27b is a course S, defined for characterizing the stress rise in the soft tissues across it, towards the bone. The distribution of von Mises stresses demonstrated sites of intensified loading in the muscular and soft connective tissues underlying the bony prominences of the pelvis. The maximal internal stresses at these sites (˜350 KPa) exceeded the interfacial compression by two orders of magnitude.

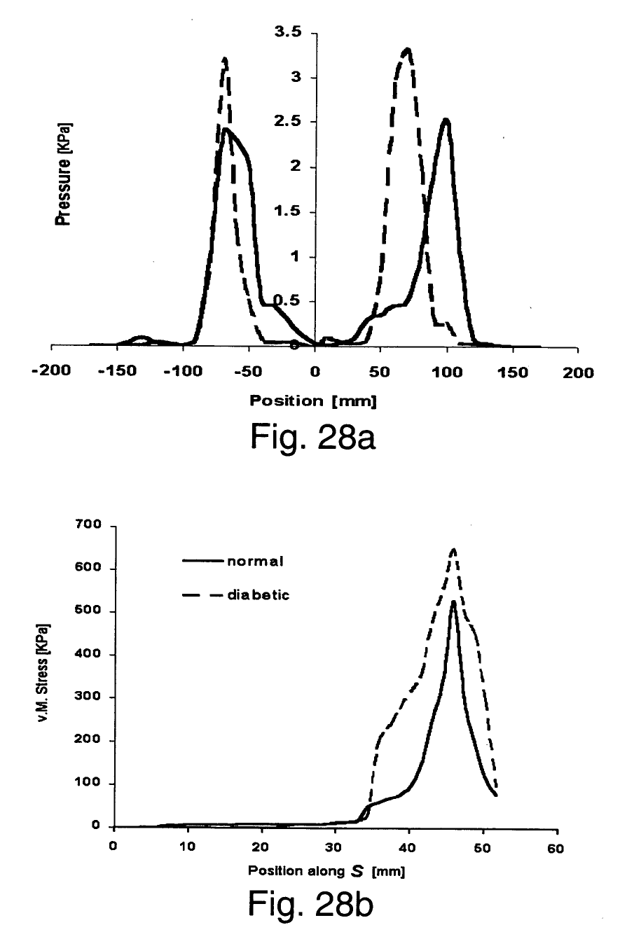

FIG. 28a and FIG. 28b show, respectively, distributions of contact compressive stress under the model's contact area with the supporting surface and the calculated internal von Mises stresses, resulting from the contact compressive stresses. The distributions of contact stress are in agreement with experimental results [Allen, V., Ryan, D. W., Murray, A. (1994) “Measurements of interface pressure between body sites and the surfaces of four specialized air mattresses”, The British Journal of Clinical Practice, 48: 125-9; Alen V., Ryan, D. W., Murray, A. (1993) “Repeatability of subject/bed interface pressure measurements”, Journal of Biomedical Engineering 15: 329-32; Brosh, T., Arcan, M. (2000) “Modeling the body/chair interaction—an integrative experimental-numerical approach”, Clinical Biomechanics 15: 217-9].

### Example 5

### Integration of In Vivo, Ex Vivo and Computation Models

In this Example, the three-dimensional anatomical study of Example 4 is extended to the case of injured muscles. It is recognized [Bosboom et al., “Quantification and Localization of Damage In Rat Muscles After Controlled Loading; A New Approach To Study the Etiology of Pressure Sores,” Medical Engineering & Physics, 23: 195-200, 2001] that exposure of muscle tissue to intensive and prolonged compression may affect its microstructure and thereby its constitutive law.

Stiffness changes of muscle tissue resulting from prolonged mechanical loading were determine and quantified, so as to facilitate the operations of risk parameter calculator 30, as generally explained hereinabove.

Experimental Methods

Mechanical properties of injured versus uninjured muscles were analyzed in the rat model. Experiments were approved by the Institutional Animal Care and Use Committee (IACUC) of Tel Aviv University and were carried out in compliance with institutional guidelines for care and use of animal models (IACUC Protocol #11-02-41).

43 male rats (weight 280±20 g) were assigned for this study. Ketamin (10 mg/Kg) and Xylazine (90 mg/Kg) were subcutaneously injected to induce anesthesia and ⅓ of this dose was injected when necessary for maintenance. Depth of anesthesia was verified by lack of pinch response.

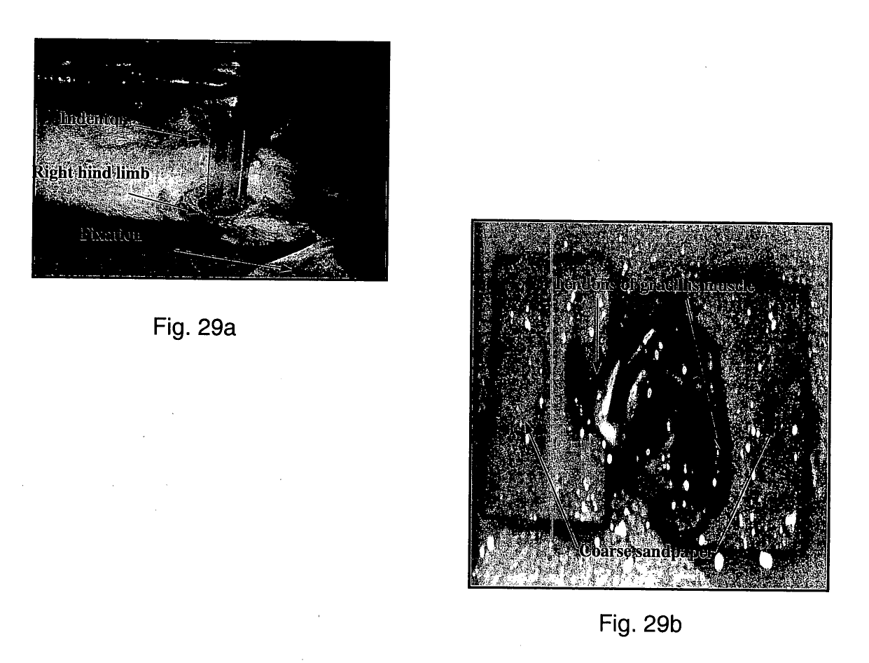

FIG. 29a is an image of a special apparatus designed for applying calibrated constant compression, which comprises a spring-derived rigid indentor 30 mm of diameter. Limb hair of the rats was carefully shaved and the rats were positioned on the apparatus. The compression was applied to the gracillis muscle of the right hind limb of sub-groups of 4-5 animals.

Using computational simulations of human recumbency (see Example 4) it was found that compression levels of 11.5 KPa, 35 KPa and 70 KPa are adequate representatives for external compression levels corresponding to the typical internal stress range in the deep human muscle layers under bony prominences. The simulations were performed under the assumption that the muscles form an isotropic and homogenous material, in which the applied external compression distributes so that a uniform internal stress is induced deep inside the muscle. It is to be understood that this assumption, which was introduced merely to simplify the simulations, is not to be interpreted as limiting.

Each of the three representative external compression levels (11.5 KPa, 35 KPa and 70 KPa) was applied for three different exposure time intervals, 2, 4, 6 hours. Thus, 9 pressure dose indices were tested (see Equation 3 above).

Once the compression was applied to all the sub-groups, an additional control sub-group was selected onto which no compression was applied. Rats of all subgroup were sacrificed by overdose of Pentobarbital and gracillis muscles were harvested from the limbs by cutting their tendons at both edges.

FIG. 29b shows a gracillis muscle positioned on a coarse sand-paper, where length, volume and weight of each muscle were recorded. Specimens were kept in saline tubes at 3° C. until mechanical testing (within no more than 30 minutes from dissection).

The excised gracillis muscles of all the groups were subjected to a comprehensive histological analysis for the purpose of revealing the effect of external compression on the microscopic structure of the muscles' tissue.

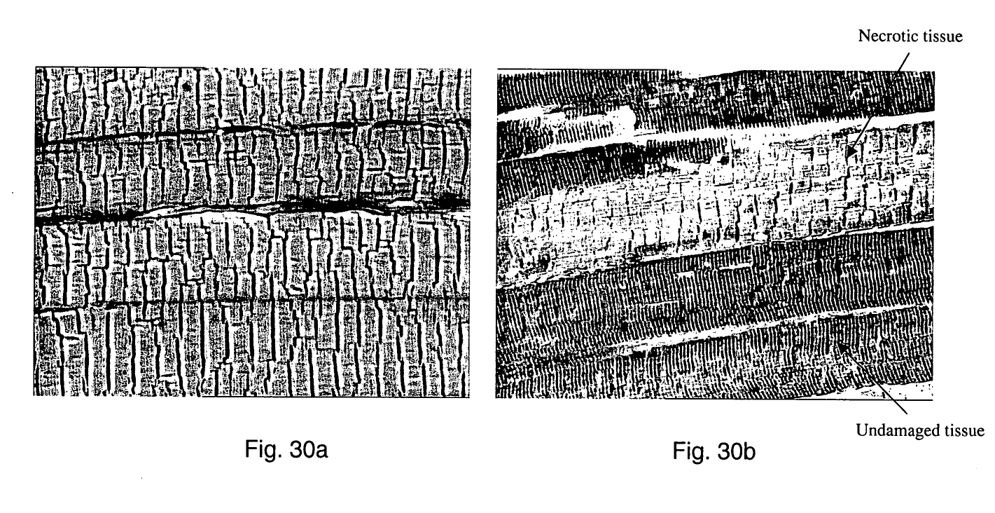

FIGS. 30a-b show representative results of histological analysis of a gracillis muscle of a rat from the control group (FIG. 30a) and a gracillis muscle of a rat from the group to which a 35 KPa external compression was applied for 2 hours (FIG. 30b). In FIG. 30b, the muscle was stained using phosphotungstic acid hematein (PTAH), were vital tissue is colored blue. As can be seen from FIG. 30a-b, the uninjured muscle has a normal cross-striation (directional preference of muscle fibrils), whereas the injured muscle experience extensive loss of cross-striation, which is associated with cell death (coagulation necrosis).

Similar results have been observed in all the muscles exposed to 35 KPa and 70 KPa for all exposure time durations. The histological findings are therefore consistent with the conclusion that exposure to external compression levels of 35 and 70 KPa for 2 hours and over, resultant in tissue necrosis.

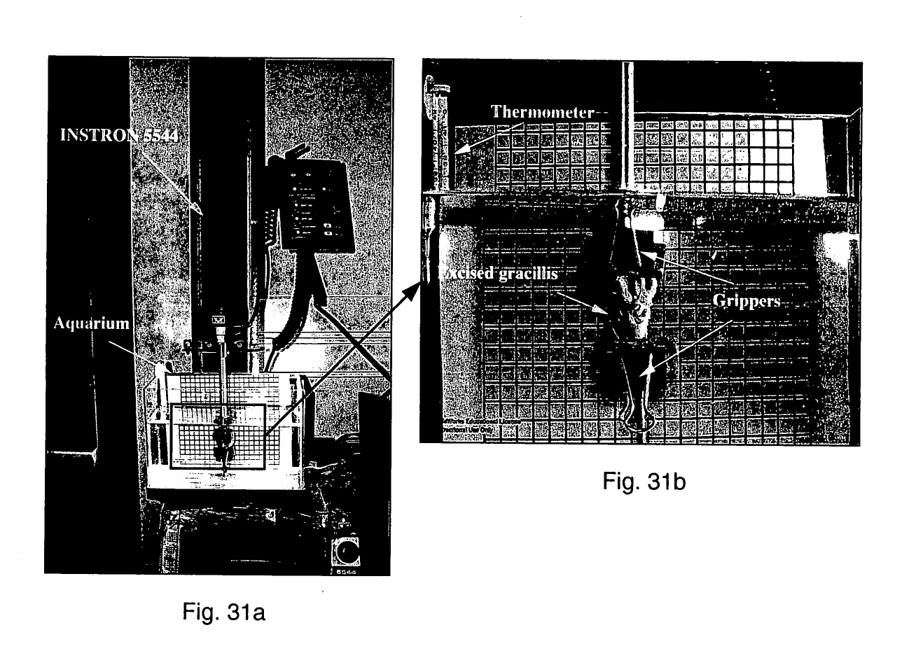

FIGS. 31a-b show the experimental setup used for ex vivo measurements of the mechanical properties of the excised muscles. Shown in FIG. 31a is an INSTRON 5544 uniaxial tension system, in which the specimens were placed in a manner that the tendons were compressed between customized jigs covered with sandpaper to prevent slipping. Enlarged image of an excised muscle positioned in the experimental setup is shown in FIG. 31b.

Tension was applied to the specimens within a transparent aquarium filled with saline at the rat's body temperature (33° C.). Load-deformation curves were obtained for the injured and uninjured (control) muscles at a rate of 1 mm/min. Deformation was visually monitored and recorded using digital and analog video systems for post-experiment slow-motion analysis to verify that muscles did not slip off the jigs.

Analysis of Experimental Data

Plots of Lagrange stress (force divided by original mean cross-sectional area) versus true strain (calculated from transient distance between jigs) were derived from the load-deformation curves. Mean cross-sectional area of muscles was obtained by dividing the muscle's volume by its unloaded length.

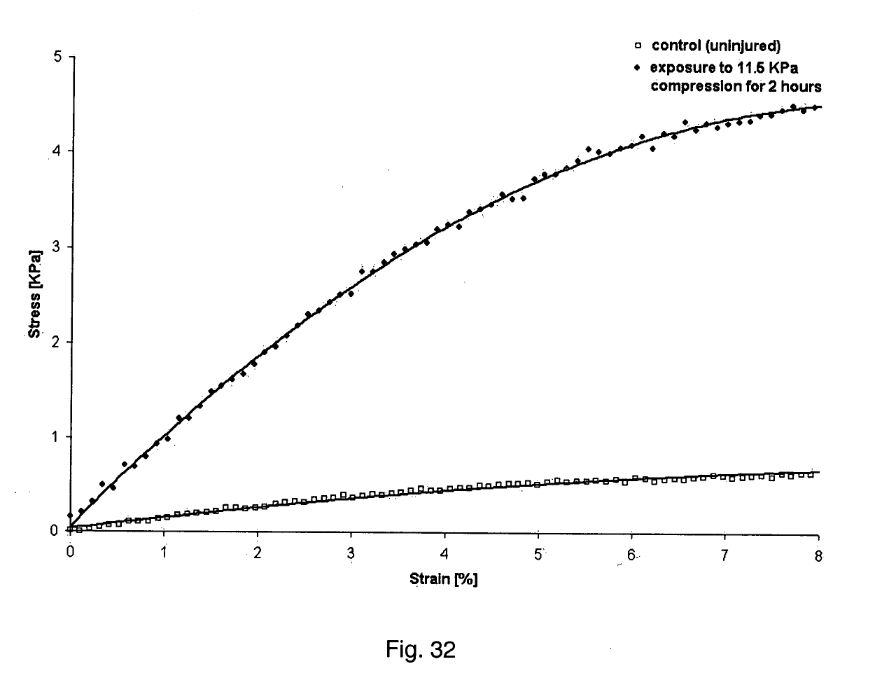

FIG. 32 shows two representative stress-strain curves. The lower curve characterizes an uninjured gracillis muscle, to which no external pressure was applied, and the upper curve characterizes an injured gracillis muscle, which was subjected to an external pressure of 11.5 KPa over a time interval of 2 hours. The stiffness difference between the muscles is vivid. For example, as can be seen from FIG. 32, a stress of less than 0.5 KPa is sufficient for creating a deformation of about 8% in the uninjured muscle, whereas the same deformation in the injured muscle can only be accomplished by a stress of about 4.5 KPa.

The significant changes in mechanical properties which are exemplified in FIG. 32 are in agreement with the histological findings (see FIGS. 30a-b), where a relatively large region of necrosis was observed. As the elasticity of the necrotic regions of the muscle is substantially smaller that the elasticity of the undamaged regions, the overall stiffness of the muscle increases with the formation of necrosis.

A low strain region was defined as the region-of-interest for the case of loading/deformation of deep muscles during recumbency. To this end, strain percentages of 2.5%, 5% and 7.5% were selected. For each strain percentage, two mechanical observables were calculated: (i) a tangent tensile modulus, Eₜ, defined as the slope of the stress-strain curve; and (ii) strain energy density, SED, defined as the area under the stress-strain curve. Thus, a total amount of six mechanical properties was used for comparison between the sub-groups.

Analysis of variance of the six mechanical properties reveled insensitivity of these mechanical properties to the above time-intervals. This insensitivity to the exposure time sets the time scale for the development of pressure sores to be below 2 hours. It is therefore assumed that during the first 2 hours, the time of exposure is a dominant factor. The analysis which followed was primarily directed at the levels of compression rather then on the exposure time, where four study groups, classified by the compression levels, were defined. Specifically, a first group is the control group (subjects to which no compression was applied), a second group included the subjects to which a compression of 11.5 KPa was applied over 2-6 hours, a third group included the subjects to which a compression of 35 KPa was applied over 2-6 hours and a fourth group included the subjects to which a compression of 70 KPa was applied over 2-6 hours.

Criteria for exclusion of specimens included apparent muscle damage caused by surgical tools during dissection, tearing of muscle adjacent to a gripper and identified slipping of specimen from the grippers. About 15% of the specimens were excluded according to these criteria.

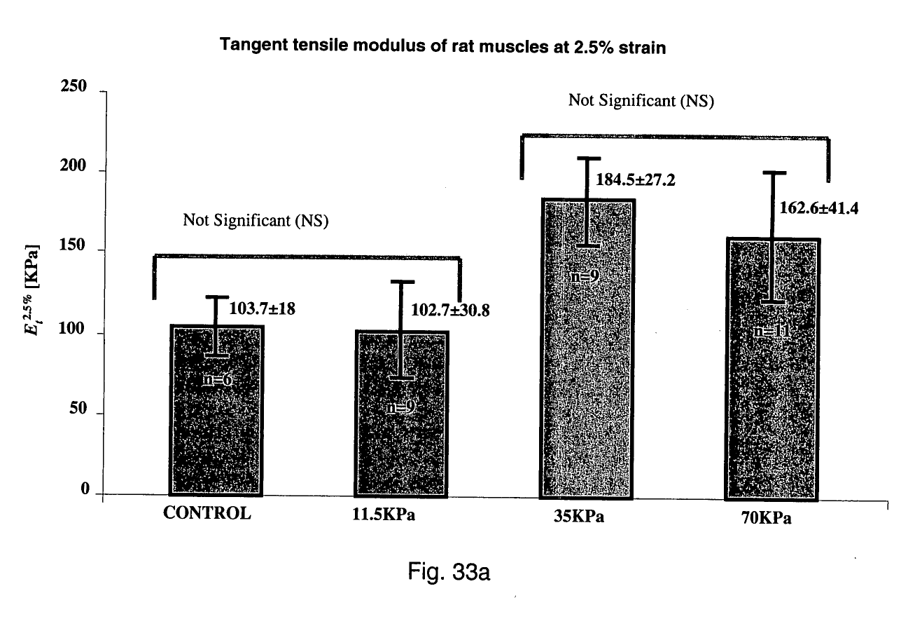

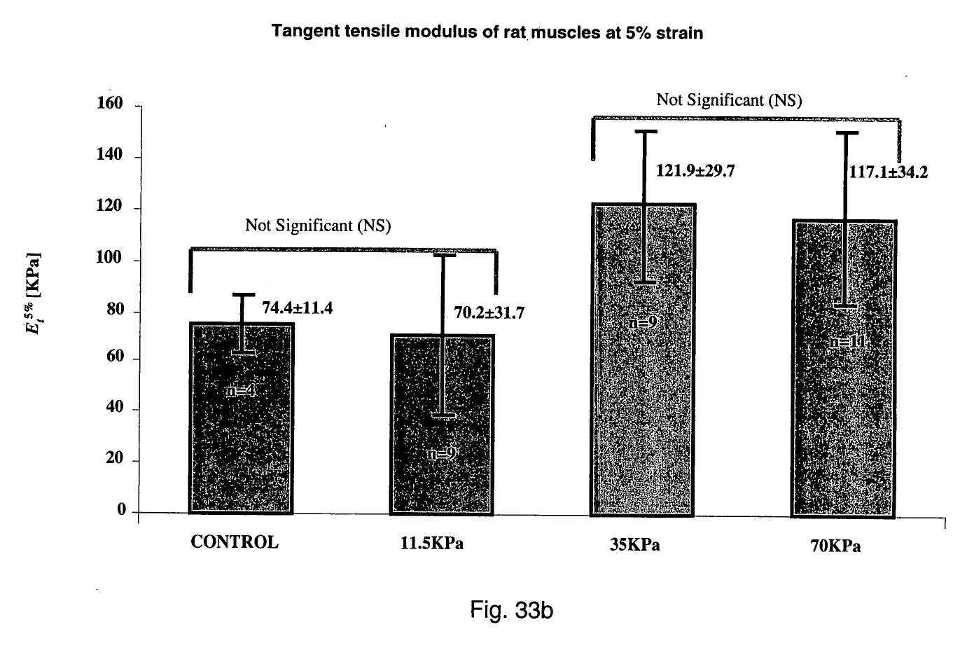

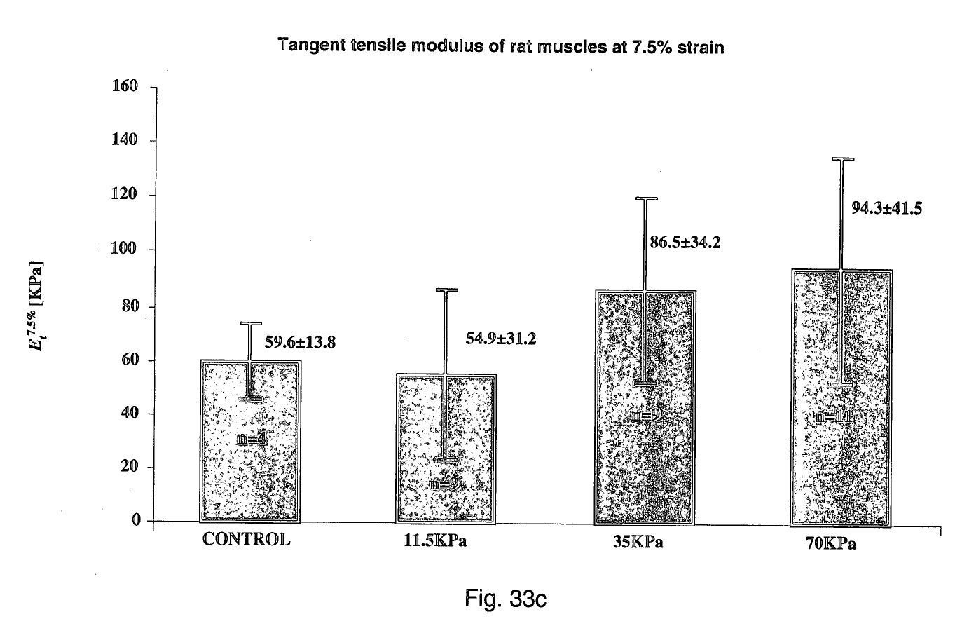

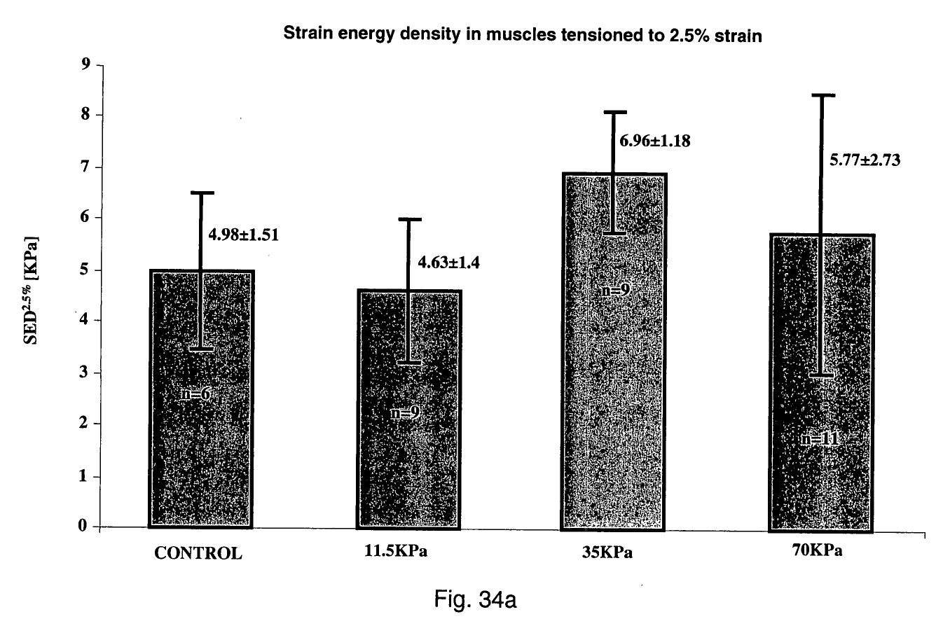

FIGS. 33a-c show tangent tensile moduli at strain percentage of 2.5% (FIG. 33a), 5% (FIG. 33b) and 7.5% (FIG. 33c), for the first, second, third and fourth groups.

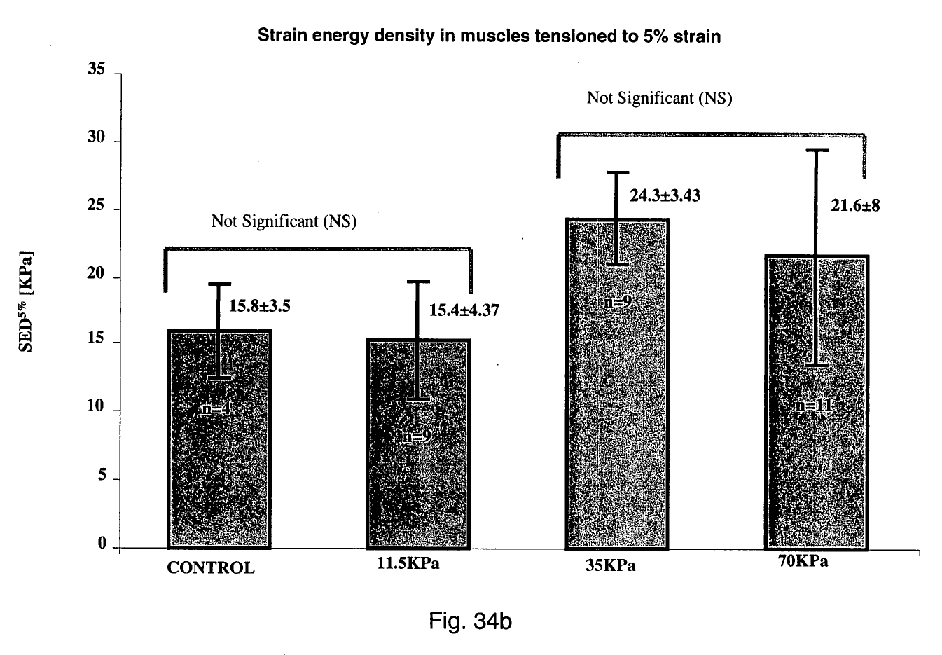

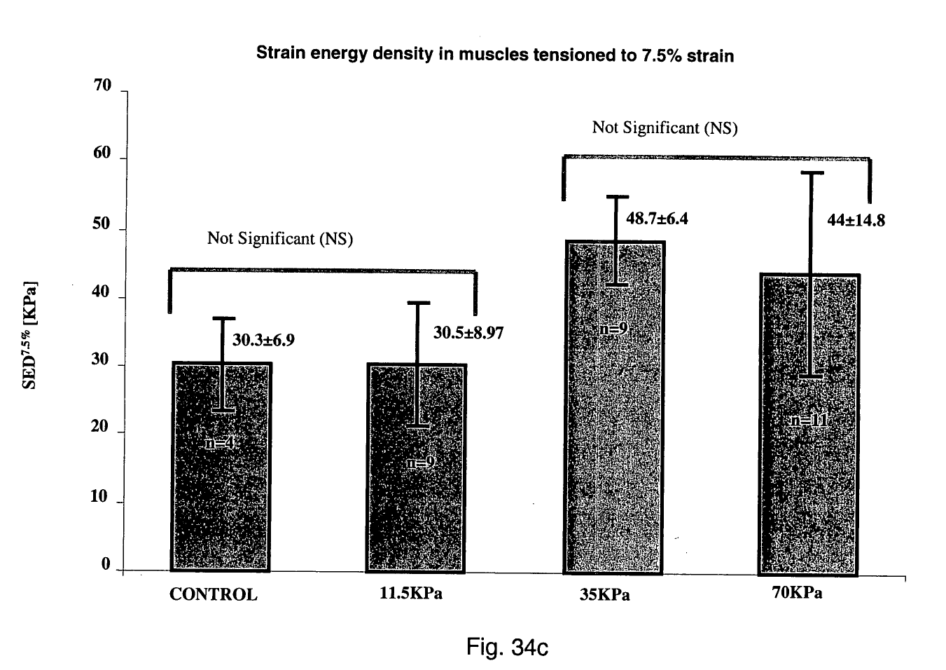

FIGS. 34a-c show strain energy densities at the above strain percentage, for the above groups. Also shown in FIGS. 33a-34c is the number, n, of non-excluded specimens in each group.

Statistical analysis, including two-way analyses of variance (ANOVA) followed by Tukey-Kramer tests, were run separately for the calculated mechanical property, to identify differences between properties of muscles subjected to different pressure doses. A p-value less than 0.05 was considered significant. In FIGS. 33a-34c, non significant differences between groups are designated by bars connecting the corresponding groups.

For two mechanical properties, E, at 7.5% strain, SED at 2.5% strain, the two-way ANOVA tests yielded no significant difference between groups. Results of the Tukey-Kramer tests for the other four mechanical properties are presented in Tables 3a-d, below.

TABLE 3a
Tukey-Kramer test for Eₜ at 2.5% strain

| | Control | 11.5 KPa | 35 KPa | 70 KPa |
|---|---|---|---|---|
| Control | — | — | — | — |
| 11.5 KPa | 1 | — | — | — |
| 35 KPa | <0.001 | <0.001 | — | — |
| 70 KPa | 0.005 | 0.001 | 0.415 | — |

TABLE 3b
Tukey-Kramer test for Eₜ at 5% strain

| | Control | 11.5 KPa | 35 KPa | 70 KPa |
|---|---|---|---|---|
| Control | — | — | — | — |
| 11.5 KPa | 0.993 | — | — | — |
| 35 KPa | 0.029 | 0.002 | — | — |
| 70 KPa | 0.047 | 0.003 | 0.976 | — |

TABLE 3c
Tukey-Kramer test for SED at 5% strain

| | Control | 11.5 KPa | 35 KPa | 70 KPa |
|---|---|---|---|---|
| Control | — | — | — | — |
| 11.5 KPa | 0.999 | — | — | — |
| 35 KPa | 0.033 | 0.01 | — | — |
| 70 KPa | 0.192 | 0.082 | 0.684 | — |

TABLE 3d
Tukey-Kramer test for SED at 7.5% strain

| | Control | 11.5 KPa | 35 KPa | 70 KPa |
|---|---|---|---|---|
| Control | — | — | — | — |
| 11.5 KPa | 1 | — | — | — |
| 35 KPa | 0.999 | 0.004 | — | — |
| 70 KPa | 0.057 | 0.029 | 0.729 | — |

As can be seen from Tables 3a-d (and FIGS. 33a-34c), consistently, across all property comparisons, the 35 KPa group and the 70 KPa group were statistically indistinguishable.

Table 4, below, summarizes values of the six mechanical properties across experimental groups after pooling data extracted from muscles subjected to 35 KPa and 70 KPa.

TABLE 4

| | Control | 11.5 KPa | 35-70 KPa |
|---|---|---|---|
| Eₜ (2.5%) | 103.7 ± 18 | 102.7 ± 30.8 | 172.5 ± 36.6 |
| Eₜ (5%) | 74.4 ± 11.4 | 70.2 ± 31.7 | 119.3 ± 31.5 |
| Eₜ (7.5%) | 59.6 ± 13.8 | 54.9 ± 31.2 | 90.8 ± 37.7 |
| SED (2.5%) | 4.98 ± 1.51 | 4.63 ± 1.40 | 6.52 ± 2.16 |
| SED (5%) | 15.87 ± 3.51 | 15.42 ± 4.37 | 22.83 ± 6.38 |
| SED (7.5%) | 30.32 ± 6.91 | 30.50 ± 8.97 | 46.12 ± 11.77 |

Table 4 demonstrates that properties of muscles subjected to 11.5 KPa compression over 2-6 hours were statistically similar to those of controls. However, muscles subjected to larger compression levels exhibited moduli and SED values that were consistently stiffer than those of normal muscles. Specifically, the tangent moduli at 2.5% and 5% strain and the SED at 5% and 7.5% strain significantly increased (typically by more than 50%) after delivery of compression of 35-70 KPa for 2-6 hour, indicating that muscle tissue became stiffer With pressure sore injury.

Compilation of the Experimental Data with a Computation Model

The experimental results demonstrate that muscle tissue undergoing prolonged pressure injury change its mechanical characteristics if a critical level of compression and exposure time is exceeded.

To further investigate whether changes in mechanical properties influence the overall pattern of stress transfer in tissues and organs around the affected sites, a 3D biomechanical model was developed, into which the compilation of the experimental data was inputted.

The biomechanical model was similar to the model presented in Example 4, and was directed at determining progression of injury, caused by exposing additional tissue volume to elevated mechanical stresses projected by the abnormally stiffened injured muscles.

Hence, the experimentally measured mechanical properties of uninjured and injured muscles were inputted into 3D geometrical models of body parts vulnerable to pressure sores.

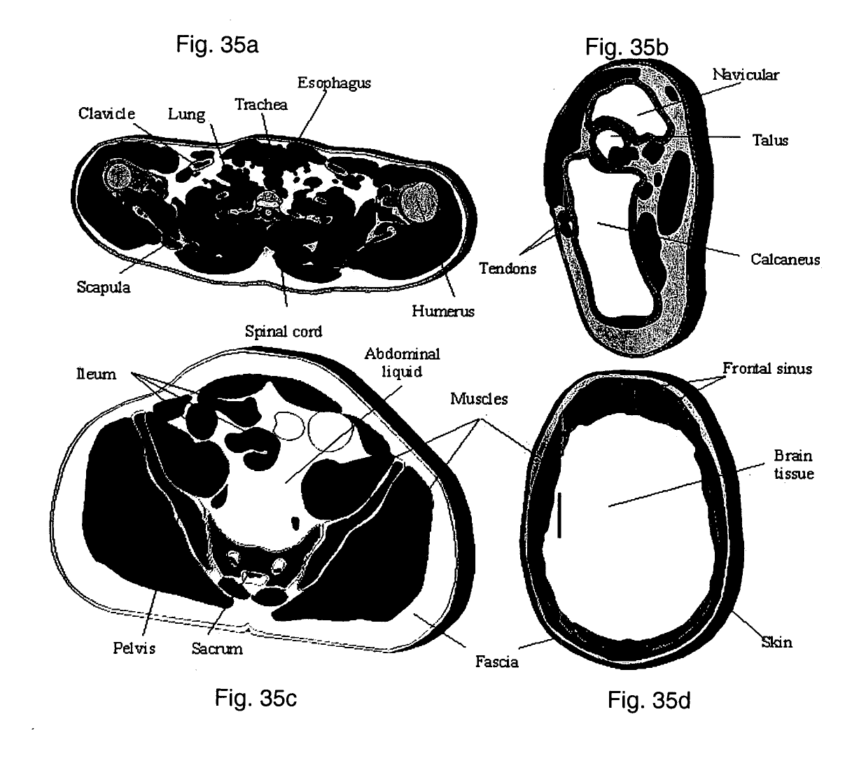

FIGS. 35a-d show 5 mm-thick anatomical slices of the mattress-supported regions of the shoulders (FIG. 35a), heels (FIG. 35b), pelvis-sacrum (FIG. 35c) and head (FIG. 35d), which were reconstructed from the Visible Human (male) digital database (ibid) in the following manner. Bones (cortical, trabecular), cartilage, muscles, colon, ileum, major blood vessels, fascia, tendons, brain tissue and skin were segmented and their contours transferred to a solid modeling software package (SolidWorks® 2003) for organ reconstructions.

Once the 3D slice geometry was obtained and the materials properties were determined, the geometry was transferred to a finite element solver, NASTRAN® 2001, for stress analysis under musculoskeletal loading during recumbency. For the purpose of structural analysis under loading, tissues were assumed to be homogenous, isotropic and non-linear elastic materials (see Example 4).

For each solid volume, the distribution of von Mises stresses (see Equation 9) was calculated.

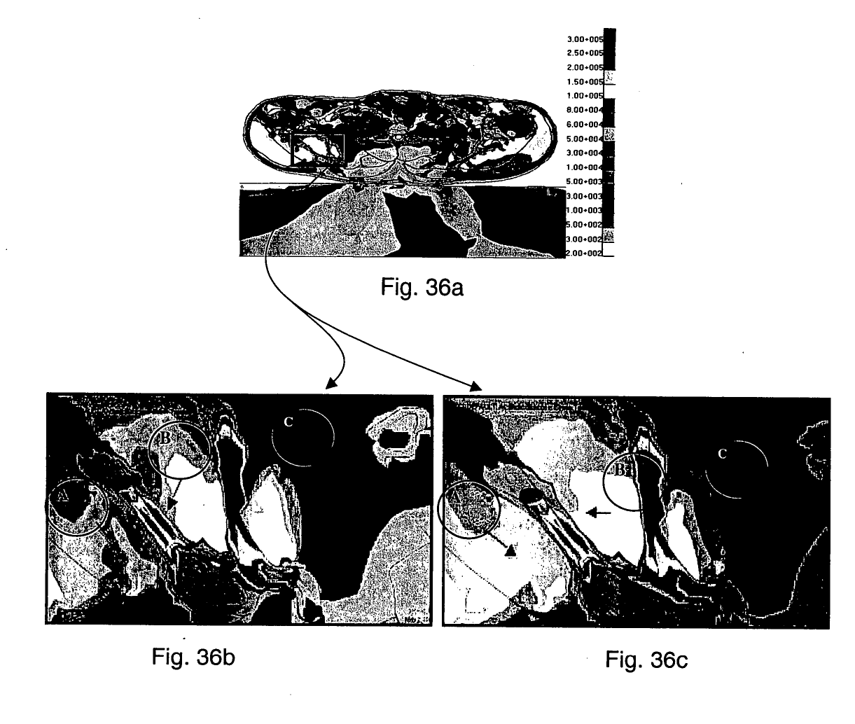

Reference is now made to FIG. 36a-c which show resulted distribution of von Mises internal stresses during recumbency within the shoulders. FIG. 36a shows the entire slice while FIGS. 36b-c show a magnified region of interest under the bony prominences, where a comparison is made between injured muscles after 2 hours of compression (FIG. 36b) and uninjured muscles (FIG. 36c). The maximal internal stresses at deep muscles generally exceeded the interfacial compression by up to two orders of magnitude. The internal stress distribution patterns during recumbency are evolving with time due to changes in the injured muscle's constitutive laws. For example, referring to sub-regions designated A, B and C in FIGS. 36b-c, stresses in muscles surrounding the scapula were predicted to increase and expand within 2 hours post-immobilization due to muscle injury and stiffening. This suggests a mechanism of deterioration in which soft tissues that were not directly affected by the intensified internal stresses could be gradually damaged due to induction of elevated stress by adjacent stiffening injured muscle tissue.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-200. (canceled)

201. A system for determining a risk of pressure ulcer onset on a subject being in contact with a supporting-surface, the system comprising:
an arrangement of interfacial stress sensors, located at predetermined locations between the supporting-surface and the subject;
an interfacial stress converter, for converting interfacial stress transmitted by said arrangement of sensors into internal stress; and
a data processor for receiving and processing data from said interfacial stress sensors and said interfacial stress converter;
said interfacial stress sensors, said interfacial stress converter and said data processor being designed and programmed for determining the risk of pressure ulcer onset on the subject being in contact with the supporting-surface.

202. The system of claim 201, wherein said sensors and said data processor are designed and programmed to alert prior to a formation, on the subject, of at least one ulcer adjacent to at least one of said sensors.

203. The system of claim 201, wherein said arrangement of sensors is designed locatable on a supporting-surface selected from the group consisting of a bed, a mattress, a chair, a wheelchair, an armchair, an operating table and a surface of a prosthesis being in contact with a residual limb.

204. The system of claim 201, wherein at least one of said interfacial stress sensors is combined with at least one additional sensor selected from the group consisting of a pressure sensor, a temperature sensor and a humidity sensor.

205. The system of claim 201, wherein said interfacial stress converter is configured to convert said interfacial stress using at least one conversion function, selected from the group consisting of a polynomial conversion function, an exponential conversion function, a rational conversion function, a power conversion function, a look-up table and any combination thereof.

206. The system of claim 205, wherein said at least one conversion function varies with at least one parameter selected from the group consisting of a weight, a body structure, an organ geometry, an age, a nutritional status, a general health condition, medications administered to the subject, a tissue mechanical property, a geometry of the supporting surface and a mechanical property of the supporting surface.

207. The system of claim 206, wherein said tissue mechanical property is selected from the group consisting of a Young modulus, a Poisson's ratio, a shear modulus, a bulk modulus, a Lamé coefficients, a tangent elastic modulus, a plurality of hyper-elastic material model invariants and a plurality of coefficients which characterize a phenomenological function describing experimental stress-strain data.

208. The system of claim 201, further comprising a risk parameter calculator for calculating a risk parameter, α, characterizing the risk of the ulcers onset.

209. The system of claim 208, wherein said risk parameter calculator comprises a first integrator for integrating said internal stress over a first predetermined period, thereby to obtain a pressure dose index, PDI.

210. The system of claim 209, wherein said risk parameter calculator comprises a pressure normalizer for normalizing said PDI, using a reference pressure dose.

211. The system of claim 202, wherein said alert is selected from the group consisting of an audio-alert, a visual-alert and a combination of an audio-alert and a visual-alert.

212. The system of claim 201, further comprising a display for displaying a risk status for each of said predetermined locations.

213. The system of claim 201, further comprising a communication channel for transmitting information from the system to a remote location.

214. The system of claim 213, wherein said remote location is a nursing control center.

215. The system of claim 213, wherein said communication channel is designed connectable to a telemetry apparatus.

216. The system of claim 213, wherein said communication channel is designed connectable to a telemedicine apparatus.

217. A method of determining a risk of pressure ulcer onset on a subject being in contact with a supporting-surface, the method comprising:
inputting information from an arrangement of interfacial stress sensors, located on predetermined locations between the supporting-surface and the subject;
converting nterfacial stress transmitted by said arrangement of sensors into internal stress; and
using said information and said internal stress for determining the risk of pressure ulcer onset on the subject being in contact with the supporting-surface.

218. The method of claim 217, further comprising alerting prior to a formation, on the subject, of at least one ulcer adjacent to at least one of said sensors.

219. The method of claim 217, wherein said arrangement of sensors is designed locatable on a supporting-surface selected from the group consisting of a bed, a mattress, a chair, a wheelchair, an armchair, an operating table and a surface of a prosthesis being in contact with a residual limb.

220. The method of claim 217, wherein at least one of said interfacial stress sensors is combined with at least one additional sensor selected from the group consisting of a pressure sensor, a temperature sensor and a humidity sensor.

221. The method of claim 217, wherein said conversion of said interfacial stress is by at least one conversion function selected from the group consisting of a polynomial conversion function, an exponential conversion function, a rational conversion function, a power conversion function, a look-up table and any combination thereof.

222. The method of claim 221, wherein said at least one conversion function is selected from the group consisting of a polynomial conversion function, an exponential conversion function, a rational conversion function, a power conversion function, a look-up table and any combination thereof.

223. The method of claim 221, wherein said at least one conversion function varies with at least one parameter selected from the group consisting of a weight, a body structure, an organ geometry, an age, a nutritional status, a general health condition, medications administered to the subject, a tissue mechanical property, a geometry of the supporting surface and a mechanical property of the supporting surface.

224. The method of claim 223, wherein said tissue mechanical property is selected from the group consisting of a Young modulus, a Poisson's ratio, a shear modulus, a bulk modulus, a Lamé coefficients, a tangent elastic modulus, a plurality of hyper-elastic material model invariants and a plurality of coefficients which characterize a phenomenological function describing experimental stress-strain data.

225. The method of claim 221, further comprising integrating said internal stress over a first predetermined period, thereby obtaining a pressure dose index, PDI.

226. The method of claim 225, further comprising normalizing said PDI, using a reference pressure dose.

227. The method of claim 218, wherein said alerting is selected from the group consisting of audio-alerting, visual-alerting and a combination of audio-alerting and visual-alerting.

228. The method of claim 217, further comprising displaying a risk status for each of said predetermined locations.

229. The method of claim 217, further comprising transmitting information to a remote location.

230. The method of claim 229, wherein said remote location is a nursing control center.

231. The method of claim 229, wherein said transmitting information is via a telemetry apparatus.

232. The method of claim 229, wherein said transmitting information is via a telemedicine apparatus.

233. A method of characterizing a risk of ulcers onset on a subject, the method comprising:
inputting interfacial stress applied on the subject;
converting said interfacial stress into internal stress, using at least one conversion function; and
using said internal stress for calculating a plurality of dose indices and a risk parameter, α, being a combination of said plurality of dose indices, thereby characterizing the risk of the ulcers onset.

234. The method of claim 233, wherein said plurality of dose indices are selected from the group consisting of a pressure dose index, PDI, a temperature dose index, TDI and a humidity dose index, HDI.

235. The method of claim 233, wherein said at least one conversion function varies with at least one parameter selected from the group consisting of a weight, a body structure, an organ geometry, an age, a nutritional status, a general health condition, medications administered to the subject, a tissue mechanical property, a geometry of the supporting surface and a mechanical property of the supporting surface.

236. The method of claim 235, wherein said tissue mechanical property is selected from the group consisting of a Young modulus, a Poisson's ratio, a shear modulus, a bulk modulus, a Lamé coefficients, a tangent elastic modulus, a plurality of hyper-elastic material model invariants and a plurality of coefficients which characterize a phenomenological function describing experimental stress-strain data.

237. The method of claim 234, further comprising integrating said internal stress over a first predetermined period, thereby obtaining said PDI.

238. The method of claim 234, further comprising integrating a temperature over a second predetermined period, thereby obtaining said TDI.

239. The method of claim 234, further comprising integrating a humidity over a third predetermined period, thereby to obtain said HDI.
